(19) United States
(12) Patent Application Publication
(10) Pub. No.: US 2009/0297579 A1
(43) Pub. Date: Dec. 3, 2009

(54) Control of Cells and Cell Multipotentiality in Three Dimensional Matrices

(75) Inventors: Carlos E. Semino, Cambridge, MA (US); Bernd Rolauffs, Brookline, MA (US); Alan Grodzinsky, Lexington, MA (US); Roger Kamm, Weston, MA (US); Elena Garreta, Barcelona (ES); Lluis Quintana, Girona (ES)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 12/227,741

(22) PCT Filed: Jun. 1, 2007
(86) PCT No.: PCT/US07/13066

§ 371 Date: Jul. 14, 2009

Related U.S. Application Data
(60) Provisional application No. 60/809,908, filed on Jun. 1, 2006.

(51) Int. Cl. A61K 9/00; C12N 5/06; A61K 45/00; A61P 43/00

(52) U.S. Cl. 424/423; 435/395; 435/373; 424/93.7

(57) ABSTRACT

Methods for wound healing or tissue regeneration by means of cell and tissue engineering, including using three-dimensional matrices with cells therein. A three-dimensional matrix, optionally containing cells such as fibroblasts, is inserted Into the wound of a subject. An anti-inflammatory factor may also be used to reduce or suppress the immune response. The wound may be covered to limit exposure to gaseous oxygen, for example, using a membrane. An anticoagulant may also be applied. In addition, cells, such as fibroblasts or stem cells, when cultured within a three-dimensional matrix, under certain conditions, can be induced to form non-fibroblast multipotent cells. When stem cells are cultured in the three-dimensional matrix, at least some of the stem cells remain as stem cells and do not differentiate. Kits for promoting the control of cells within three-dimensional matrices are also disclosed.

### FIELD OF INVENTION

The present invention generally relates to cell and tissue engineering and, in particular, to cells within three-dimensional matrices and uses thereof, for example, for wound healing or tissue regeneration.

### BACKGROUND

The extracellular matrix (ECM) is a vital component of cellular microenvironments, providing cells or tissues with the appropriate architecture for normal growth and development. The extracellular matrix includes glycoproteins such as collagen, other proteins such as fibrin and elastin, minerals such as hydroxyapatite, fluids such as blood plasma or serum, etc. The extracellular matrix also provides support and anchorage for the cells, providing a way of separating the tissues, and regulating intercellular communication. Additionally, the extracellular matrix has also been implicated in influencing and enabling cell proliferation, differentiation, and proper cell-cell and cell-tissue interactions.

Most cell culture and cell signaling research has used two-dimensional surfaces to culture and study cells, for example, in Petri dishes, flasks, or microwell plates. Often, factors such as various chemicals or hormones are added to the cell culture. However, less work has been done towards recreating a three-dimensional cell culture environment.

### SUMMARY OF THE INVENTION

The present invention generally relates to cells within three-dimensional matrices and uses thereof, for example, for wound healing or tissue regeneration. The subject matter of the present invention involves, in some cases, interrelated products, alternative solutions to a particular problem, and/or a plurality of different uses of one or more systems and/or articles.

In one aspect, the present invention is a method. The method, according to one set of embodiments, includes an act of culturing fibroblasts within a three-dimensional matrix in the presence of an anti-inflammatory factor. The matrix may include, for example, one or more of a peptide scaffold, a peptide hydrogel, a self-assembled peptide, a polysaccharide, agarose, alginate, Collagen I, hyaluronate, nanofibers, and/or a repeating peptide sequence, such as RADA, as well as other materials, such as those described herein. In some cases, the fibroblasts are cultured under conditions such that at least some of the fibroblasts form non-fibroblast multipotent cells.

In accordance with another set of embodiments, the method includes an act of culturing fibroblasts within a three-dimensional matrix under conditions such that at least some of the fibroblasts form non-fibroblast multipotent cells. The method may be performed, e.g., in vitro or in vivo. In some cases, at least some of the non-fibroblast cells can be isolated from the 3-dimensional matrix, and in some embodiments, at least some of the non-fibroblast cells are able to differentiate into more than one type of cell, and/or at least some of the non-fibroblast multipotent cells are able to differentiate into more than one cell type. As an example, in one embodiment, at least some of the non-fibroblast cells are osteoblast-like cells. In some instances, at least some of the non-fibroblast cells form a mineralized matrix, and in certain cases, at least some of the non-fibroblast cells can express alkaline phosphatase activity, collagen I, intracellular osteopontin, and/or transcription factor Runx2.

The method, in yet another set of embodiments, includes an act of culturing fibroblasts within a three-dimensional matrix under conditions such that at least some of the fibroblasts form non-fibroblast multipotent cells. In one embodiment, at least some of the non-fibroblast multipotent cells are implanted into a subject.

In still another set of embodiments, the method includes an act of inserting, into a wound of a subject, a three-dimensional matrix containing an anti-inflammatory factor. The wound may be, for instance, a skin wound, a burn, a severed digit, etc., as described below. In some cases, the wound may be limited to exposure to gaseous oxygen, for instance, by applying a membrane at least substantially impermeable to oxygen to at least a portion of the wound. The three-dimensional matrix, in some embodiments, can be seeded with cells, such as fibroblasts. In some cases, the fibroblasts are isolated from the subject having the wound. In addition, in certain instances, an anticoagulant is applied to the subject.

In yet another set of embodiments, the method includes acts of inserting, into a wound, a three-dimensional matrix, and suppressing an immune response within the wound. In some cases, the three-dimensional matrix may be immobilized relative to the wound, for instance, with a clamp.

In one set of embodiments, the method includes acts of cauterizing at least one artery within a wound, and inserting, into the wound, a three-dimensional matrix.

The method, according to another set of embodiments, includes acts of removing a tissue comprising fibroblasts from a subject, extracting fibroblasts from the tissue, adding the fibroblasts to a three-dimensional matrix, and implanting the three-dimensional matrix into the subject. The wound may be, e.g., a skin wound, a burn, a severed digit, or the like. The fibroblasts may be implanted into the subject within 1 day after removal of the tissue from the subject, and optionally, the fibroblasts can be grown within the three-dimensional matrix for at least about a week. In some cases, the three-dimensional matrix may be implanted into a wound of the subject.

In still another set of embodiments, the method includes acts of implanting fibroblasts into a wound, and suppressing an immune response within the wound.

The method, in another set of embodiments, includes acts of culturing stem cells in a three-dimensional matrix for at least 7 days in media substantially free of stem cell promoting factors; and thereafter, identifying at least some of the cells as stem cells. For example, at least some of the cells may be identified using an Oct4 expression assay. In some cases, at least some of the stem cells may form osteoblast-like cells. In one set of embodiments, at least some of the stem cells may be isolated from the 3-dimensional matrix after culturing the cells. In some cases, the method also includes an act of causing at least some of the stem cells to form a differentiated tissue. In one embodiment, the stem cells may be identified within the differentiated tissue.

In one set of embodiments, the method is a method of regenerating tissue. The method may include acts of applying a three-dimensional matrix to a severed tissue, the matrix comprising one or more regeneration factors, and reducing exposure of the severed tissue to oxygen to promote regeneration of the tissue. The tissue may be, for example, a severed digit, severed by a surgical procedure, or the like, as discussed below.

The present invention, in another aspect, is an article. In one set of embodiments, the article includes a culture comprising fibroblasts. In some cases, the culture also includes an anti-inflammatory factor. The culture can be, e.g., in vitro or in vivo. At least some of the fibroblasts may be located within a three-dimensional matrix, in certain embodiments.

The article, according to another set of embodiments, includes a three-dimensional matrix containing an anti-inflammatory factor. In some cases, the three-dimensional matrix comprises fibroblasts.

In yet another aspect, the present invention includes methods and related protocols for culturing cells or stem cells (e.g., dermal fibroblasts) of embryonic or adult origin within a three-dimensional matrix, such as a three-dimensional nanofiber matrix, to obtain cells with mesenchymal multipotential capacity in vitro.

The present invention, in still another aspect, includes methods and related protocols for culturing cells or stem cells (in particular dermal fibroblasts) of embryonic or adult origin within a three-dimensional matrix, such as a three-dimensional nanofiber matrix, to obtain cells with multipotential capacity to differentiate into one or more mesenchymal tissue-type, optionally with the property of engaging in redevelopment programs and self-organization. In some cases, the cells may undergo a process that “recapitulates” various states of development, including induction and expression of significant markers such as transcription factors and molecules (proteins, lipids, polysaccharides, etc.) that contribute to the appropriate differentiated phenotype. In certain instances, the markers may have a pattern of expression that present positioning in the organizing structure, for example, induction and/or expression of significant developmental markers that resemble body plan and segmentation in the organized structure.

In another aspect, the present invention is directed to a method of making one or more of the embodiments described herein. In another aspect, the present invention is directed to a method of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B are schematic flowcharts illustrating certain embodiments of the invention;

FIG. 1C is a scanning electron photomicrograph illustrating a matrix formed from a self-assembling peptide, useful in some embodiments of the invention;

FIGS. 2A-2D illustrate GFP (green fluorescent protein) expression in mESCs (mouse embryonic stem cells), according to one embodiment of the invention;

FIGS. 3A-3D illustrate certain EB (embryoid body) derived cells in another embodiment of the invention;

FIGS. 4A-4C illustrate certain three-dimensional cultures, in yet another embodiment of the invention;

FIGS. 5A-5D illustrate certain MEFs (mouse embryonic fibroblasts) in still another embodiment of the invention;

FIG. 6 schematically illustrates a three-dimensional matrices used to treat a wound, according to one embodiment of the invention;

FIG. 7 illustrates cell dedifferentiation, according to another embodiment of the invention;

FIGS. 8A-8H illustrate stained certain MEFs, in yet another embodiment of the invention;

FIGS. 9A-9B are graphs illustrating expression of nestin and osteopontin of various cultured fibroblasts, according to one embodiment of the invention;

FIGS. 10A-10B illustrate chondrogenesis and adipogenesis of MEFs, according to another embodiment of the invention;

FIGS. 11A-11C illustrate the morphological appearance of MEF cultures, according to another embodiment of the invention;

FIG. 12 illustrates a phenotype assessment under different culture conditions, in yet another embodiment of the invention;

FIGS. 13A-13D shows morphogenesis of MEF in soft self-assembling peptide cultures, in still another embodiment of the invention;

FIGS. 14A-14B illustrates brachyury expression in a MEF culture, in yet another embodiment of the invention;

FIGS. 15A-15C illustrates brachyury expression in a MEF culture, in still another embodiment of the invention; and

FIGS. 16A-16C shows molecular characterization of the mesodermal induction process observed in MEFs cultured in RAD16-I cultures, according to another embodiment of the invention.

FIG. 17 shows Quantitative Real Time PCR(RT-PCR) of Sox9 transcription factor.

FIGS. 18A-18B shows inhibition of the 3D-bilateral structure development by cell cycle arrest induced by staurosporine.

### BRIEF DESCRIPTION OF THE SEQUENCES

SEQ ID NO: 1 is RADARADARADARADA, a repeating peptide sequence;

SEQ ID NO: 2 is (AEAEAKAK)₂, a repeating peptide sequence; and

SEQ ID NO: 3 is (ARARADAD)₂, a repeating peptide sequence.

SEQ ID NO: 4 is CATGTACTCTTTCTTGCTGG a forward primer sequence.

SEQ ID NO: 5 is GGTCTCGGGAAAGCAGTGGC a reverse primer sequence.

### DETAILED DESCRIPTION

The present invention generally relates to cell and tissue engineering and, in particular, to cells within three-dimensional matrices and uses thereof, for example, for wound healing or tissue regeneration. One aspect of the invention is generally directed to methods of wound healing or tissue regeneration. In some embodiments, a three-dimensional matrix, optionally containing cells such as fibroblasts, is inserted into the wound of a subject. An anti-inflammatory factor may also be used in certain cases to reduce or suppress the immune response. In some instances, the wound may be covered to limit exposure to gaseous oxygen, for example, using a membrane. In one set of embodiments, an anticoagulant is also applied. Another aspect of the invention is generally directed to culturing cells, such as fibroblasts or stem cells, within a three-dimensional matrix. In some cases, fibroblasts within a three-dimensional matrix, under certain conditions, can be induced to form non-fibroblast multipotent cells. In certain embodiments, stem cells are cultured in the three-dimensional matrix such that at least some of the stem cells remain as stem cells and do not differentiate, even, in some cases, where the media is substantially free of stem cell promoting factors. Yet other aspects of the invention are directed to kits or methods of promoting the control of cells within three-dimensional matrices.

Cellular self-organization studies have been mainly focused on models such as Volvox, the slime mold Dictyostelium discoideum, and animal (metazoan) embryos. Interestingly, these models have something in common: their individual cells need to adhere together to form a cohesive organism. Free Dictyostelium cells synthesize a sticky 24-kDa glycoprotein under nutritional deficit, becoming increasingly adhesive and promoting the formation of cellular aggregate that undergo differentiation into an organized structure. (Raper KB (1940) Pseudoplasmodium formation and organization of Dictyostelium discoideum. J Elisha Mitchell Sci Soc 56: 241-282. Knecht D A, Fuller D, Loomis W F (1987) Surface glycoprotein gp24 involved in early adhesion of Dictyostelium discoideum. Dev Biol 121: 277-283.) Similar mechanisms occur in early animal embryos where cells adhere together to form the tissues and organs during development. Also animal tissues undergoing regeneration present intrinsic properties of embryonic systems including cell multipotential capacity, pattern expression of developmental genes by a self-organization process to rebuild tissue complexity and function. For instance, the process of mammal digit tip regeneration displays phases similar to those found in limb regeneration in amphibians: (1) Apical Epithelial Cap (AEC) formation (2) blastema-like formation by dermal fibroblast and myotube dedifferentiation, and (3) regeneration or re-development, leading to scar-less wound healing. It has been discovered according to the invention that the recreation of a suitable microenvironment similar to that of regenerative areas (with reduced or non-inflammatory response) can be developed in vitro by recreating the biological, biophysical and biomechanical conditions that evoke the intrinsic capacity of adult tissues to proceed to regeneration instead of scarring. An in vitro system has been developed that resembles some aspects of a regenerative blastema, where dermal fibroblasts (and other differentiated cells) not only acquire properties such as cell dedifferentiation and multipotentiality but that, as a whole, engage in a re-development program manifested by spontaneous pattern formation by a self-organization process. The in vitro cellular system described herein undergoes a process that resembles many aspects of animal development including cell aggregation, proliferation, migration and tissue specification but most importantly: morphogenesis and pattern formation. Thus, the methods promote self-organization into naturally occurring-like structures, i.e., mimicking natural tissue.

When primary mouse embryonic fibroblasts are cultured in a soft nanofiber scaffold they establish a cellular network that causes an organized cell contraction, proliferation and migration that ends in the formation of a symmetrically bilateral structure with a distinct central axis. Strikingly, the 3D-bilateral structure up-regulated the expression of the mesodermal organizer gene Brachyury localized first in a line of cells along the central axis and extending then to both sides of the structure. The subset of chondrogenesis and pre-osteogenesis transcription factors Sox9 and Runx2, respectively, were upregulated at around the same time. This was followed by, development of cartilage-like tissue at both sides of the central axis was evidenced by the synthesis of glycosaminoglycans and collagen type II, with a pattern that resembles an early paraxial mesoderm. Staurosporine treatment prevented the formation of the 3D-bilateral structure indicating that proliferation is important for the development of the structure. The invention provides a novel experimental system of cellular self-organization that develops into a patterned bilateral structure.

The invention is based at least in part on the discovery that the 3 dimensional environment in which a cell is grown, regardless of the chemical nature of the environment (self-assembling peptide nanofiber, polysaccharide or protein matrix), is sufficient to promote dedifferentiation of fibroblasts into a multipotent mesenchymal progenitor-like cell. Thus, it is believed that the cells “sense” the environment and are reprogramed into a multipotent progenitor.

The fibroblasts underwent spontaneous adipogenesis in 3 dimensions regardless of the other conditions. However, 3 dimensional matrix composed of self-assembling peptide caused fibroblasts to begin a default chondrogenic differentiation process, presumably by creating a special cell microenvironment, whereas other tested 3 dimensional matrices did not push the cells to this state under the tested conditions. This could be due to the chemical differences between these three scaffolds. The polysaccharide (agarose) has shown low interaction with fibroblasts as well as poor contracting capacity: cells adopt a spherical shape and have very little movement inside the scaffold. Collagen I is the natural extracellular matrix component of the dermis, ensuring high interaction between the cells and the matrix. Furthermore, this material is instructive in guiding embryonic fibroblasts into this lineage, preventing them from spontaneous differentiation into a chondrogenic lineage. Finally, self-assembling peptide scaffolds lack inherent signaling capacity per se suggesting that the system could naturally undergo chondrogenic lineage differentiation. Not only does the self-assembling peptide allow spontaneous chondrogenesis, but it also promotes, in certain conditions, a unique in vitro cellular self-organization that resembles early embryonic stages. Furthermore, similarly to what has been observed in animal development, the progression of this morphogenesis is dependent on proliferation as evidenced by the effects of Staurosporine the system contraction. This phenomenon seems to have a close relationship to the spontaneous chondrogenic differentiation of MEFs. Such localized natural chondrogenic induction may be the result of mesenchymal progenitor condensation and differentiation under the control of an organized process that directs patterned cell differentiation.

Thus, the system described herein undergoes organized contraction developing into a bilateral shape structure with a central axis, called 3D-bilateral structure, resembling an early vertebrate embryonic stage. These remarkable morphological changes suggest that the system might be engaging in a cellular self-organization process. The initial conditions that were provided to the cells (3D-culture of MEFs in self-assembling peptide scaffolds) recapitulates cellular and morphological changes of tissues undergoing development (proliferation, migration and condensation) and the expression pattern of early embryonic genes (organizers). Moreover, the process ends in the production of specific tissues such as patterned paraxial cartilage-like tissue. Most importantly, a group of differentiated cells under special “environmental conditions” can proceed to a self-organization process, characteristic of systems undergoing development. These discoveries suggest important utilities for the methods of the invention, in wound healing tissue generation and regeneration, stem cell studies and other therapeutic uses. The system can be used in some embodiments to culture cells carrying specific mutations or transgenic genes and to obtain 3D-bilateral structures that recreate certain morphogenetic processes without the use of pregnant females and their embryos.

In certain aspects of the invention, cells, such as fibroblasts or stem cells, are cultured using a matrix for relatively long periods of the time, for example, days to weeks. As used herein, a “matrix” is a material, typically organic or biologically-derived, that cells can bind to, i.e., the matrix includes a cytophilic material. The matrix may be a two-dimensional (“2D”) matrix, i.e., a surface upon which cells can be cultured, but do not significantly penetrate, or a three-dimensional (“3D”) matrix, i.e., a material that surrounds cells, within which cells can be cultured. In some embodiments, a three-dimensional matrix is particularly useful. A three-dimensional matrix is often porous, such that nutrients, gases, waste products, and/or other materials can pass through the three-dimensional matrix. In some cases, the pores of the three-dimensional matrix may be sufficiently large such that cells can migrate into or through the three-dimensional matrix.

In some embodiments, the material defining the matrix comprises a polymer, for instance, a polysaccharide and/or a peptide, such as a repeating peptide. In certain cases, the matrix is formed by a series of nanofibers, e.g., nanofibers of polysaccharides and/or peptides, which together define a porous matrix having interstitial spaces through which materials or cells can pass. The matrix may also be designed to have certain properties. For example, the matrix may be designed to be a gel or a hydrogel, biodegradable, or the matrix may be designed to be self-assembling. In some instances, the matrix is designed to be a low signaling matrix, for example, having low instructive capacity with respect to cells on or within the matrix. As described in greater detail below, a low signaling matrix is a matrix which generally does not contain significant amounts of ligands which can alter cell behavior.

In one set of embodiments, the material defining the matrix comprises a polysaccharide, i.e., a polymer formed of carbohydrates or other sugar or saccharide moieties (or other similar moieties), and/or derivatives thereof. Non-limiting examples of polysaccharides that can be present within the matrix include agrose, alginate, hyaluronate, Collagen I, polylactic acid, polyglycolic acid, etc. Polysaccharides are generally derived from carbohydrates or other sugar moieties using polymerization techniques well-known to those of ordinary skill in the art, e.g., by reduction of carbonyl groups, by oxidation of one or more terminal groups to carboxylic acids, by replacement of one or more hydroxy group(s) by a hydrogen atom, an amino group, a thiol group, or similar heteroatomic groups, etc. Typically, the polysaccharide comprises many repeating units of carbohydrates or other sugar moieties joined together, for example, glycosidic linkages. The polysaccharide may be linear, or branched in certain instances. In some cases, the polysaccharide may be multimeric, i.e., comprising more than one type of polymer chain, and/or the polysaccharide may be a copolymer, i.e., comprising more than one type of repeat unit.

In another set of embodiments, the material defining the matrix comprises a peptide, i.e., comprising one or more amino acids. Those of ordinary skill in the art will be aware of amino acids commonly used to form peptides, for example, the 20 amino acids commonly found in nature, typically in the L-isomer, i.e., alanine, arginine, asparagine, aspartic acid, cysteine, glutamine, glutamic acid, glycine, histidine, isoleucine, leucine, lysine, methionine, phenylalaine, proline, serine, threonine, tryptophan, tyrosine, and valine. As used herein, a peptide is not limited by its length, or the number of amino acids that form the peptide, e.g., the peptide may include tens, hundreds, or thousands of amino acids. In some cases, various peptides are polymerized or otherwise reacted together (e.g., covalently) to form the material defining the matrix.

In some embodiments, the peptide comprises a repeating sequence. The repeating sequence may be used, for example, to promote self-assembly, or to provide a low signaling environment, as discussed below. It should also be noted that the peptide may contain, in some cases, other sequences besides the repeating sequence. Such a peptide comprising a repeating sequence is referred to herein as a “repeating peptide.” The repeating sequence is generally a fairly short sequence, for example, less than 16 amino acids, less than 15 amino acids, less than 14 amino acids, less than 13 amino acids, less than 12 amino acids, less than 11 amino acids, less than 10 amino acids, less than 9 amino acids, less than 8 amino acids, less than 7 amino acids, less than 6 amino acids, less than 5 amino acids, less than 4 amino acids, etc., that is contiguously repeated within the peptide any number of times. For example, the repeating unit may be repeated within the peptide at least 3 times, at least 4 times, at least 5 times, at least 6, times, at least 7 times, at least 10 times, at least 25 times, at least 50 times, etc. One non-limiting example of a repeating peptide is AcN-RADARADARADARADA-CONH₂ (SEQ ID NO: 1), in which the repeating sequence “RADA” is repeated 4 times within the peptide. Other non-limiting examples include (AEAEAKAK)₂ (SEQ ID NO: 2) or (ARARADAD)₂ (SEQ ID NO: 3). Such a repeating peptide may then be polymerized or otherwise reacted to form the material defining the matrix.

In certain embodiments, at least a portion of the material defining a matrix can be a self-assembling material, i.e., a material that, under certain conditions, spontaneously aggregates to form a defined structure (i.e., not a random aggregate). In some cases, the self-assembling materials may spontaneously assemble to from a matrix under ambient conditions, for example, when in solution. In other cases, the self-assembling materials may self-assemble to from a matrix when a certain condition is met, for example, when a certain temperature is reached, when the anionic strength of a solution containing the self-assembling materials is increased, when the pH of the solution is raised or lowered to a certain value, etc. As an example, a solution may contain certain peptides, such as repeating peptides, that are able to spontaneously aggregate to form a three-dimensional matrix. A specific non-limiting example of such a self-assembling peptide is AcN-RADARADARADARADA-CONH₂ (SEQ ID NO: 1). Other examples of self-assembling peptides include (AEAEAKAK)₂ (SEQ ID NO: 2) and (ARARADAD)₂ (SEQ ID NO: 3). Additional self-assembling peptides are described in more detail in, for example, U.S. Pat. Apl. Pub. No. 2005/0181973 by Genové, et al., published Aug. 18, 2005; U.S. Pat. No. 5,670,483 by Zhang, et al., issued Sep. 23, 1997; U.S. Pat. No. 5,955,343 by Holmes, et al., issued Sep. 21, 1999; U.S. Pat. No. 6,548,630 by Zhang, et al., issued Apr. 15, 2003; or U.S. Pat. No. 6,800,481 by Holmes, et al., issued Oct. 5, 2004, each incorporated herein by reference.

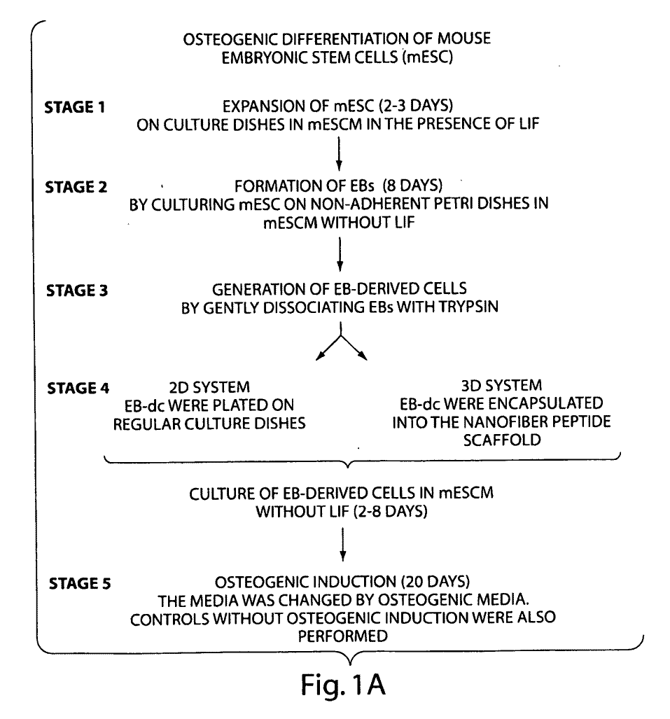

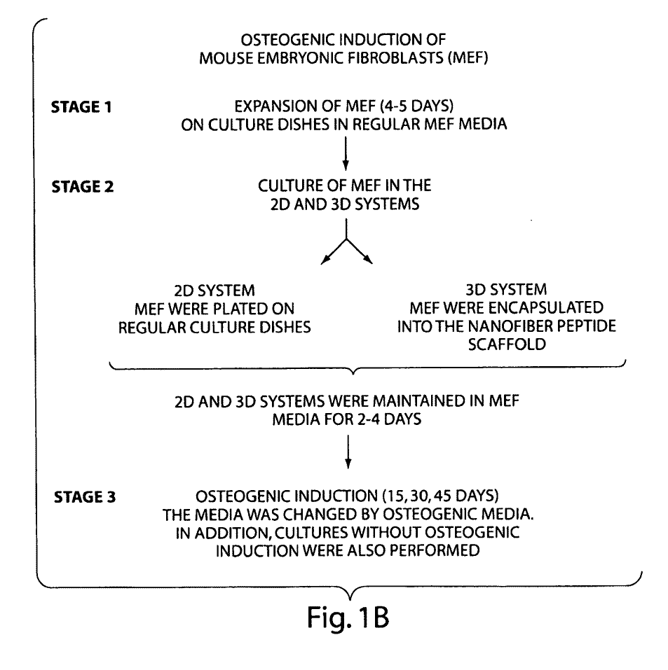

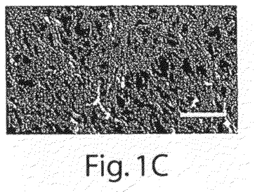

In some embodiments of the invention, the material defining the matrix may comprise one or more nanofibers, e.g., synthetic fibers, nanofibers of polysaccharides and/or proteins and/or peptides, or the like. In some cases, the nanofibers may be formed from a self-assembling peptide, such as those described herein. Within a three-dimensional nanofiber matrix, cells can be contained within the spaces between the nanofibers, thus, the nanofibers may act as an extracellular matrix for the cells. The nanofibers of a matrix typically have a characteristic or average diameter on the order of nanometers, i.e., less than about one micrometer. For instance, the average diameter of the nanofibers may be between about 1 nm and about 1,000 nm, between about 5 nm and about 500 nm, between about 10 nm and about 100 nm, etc. In some embodiments, the fibers may have an average diameter of less than about 100 nm, or less than about 30 nm. The spaces between the nanofibers may also vary, i.e., the density of nanofibers within the matrix. For instance, the spaces between the nanofibers may be between about 1 nm and about 500 nm, between about 5 nm and about 200 nm, etc. The size of the nanofibers, and the spacing between the nanofibers, may be selected as desired by those of ordinary skill in the art, depending on a particular application (e.g., depending on a particular cell type to be used within the nanofiber matrix). The nanofibers within the matrix may be the same, and/or have a range of different compositions and/or sizes or diameters, and in some cases, the diameter of the fiber may vary with respect to the length of the fiber. An example of a nanofiber structure used in conjunction with certain embodiments of the invention is shown in FIG. 1C.

The material defining the matrix, in certain embodiments of the invention, may also be selected to be a gel, such as a hydrogel. Generally speaking, a hydrogel is a gel material that is able to expand or swell in the presence of water, which becomes physically incorporated within the hydrogel. Expansion of a three-dimensional matrix may provide more space for cells to become incorporated within the matrix. Those of ordinary skill in the art will be familiar with gels and hydrogels, and properties of gels and hydrogels. Large amounts of water may be incorporated within a hydrogel in some cases. For example, after swelling, water may constitute at least about 10% of the total weight (or volume) of a hydrogel, and in some cases, at least about 20%, at least about 30%, at least about 40%, at least about 50%, at least about 60%, at least about 70%, at least about 80%, at least about 90%, or at least about 99% of the total weight (or volume). The water incorporated within the hydrogel, of course, may include other materials. For example, a hydrogel may be exposed to cell culture media in order to swell the hydrogel using the media, i.e., water and salts, buffers, nutrients, dissolved gases, etc. may become incorporated within the hydrogel matrix.

In some embodiments, the material defining the matrix can be selected to be biodegradable, i.e., the material is able to degrade, either partially or completely, when exposed to prolonged contact with a biological system, for example, when placed in contact with cell culture or with a subject, e.g., in an implant. In some cases, the material may degrade upon contact with water. For instance, if the material comprises a polymer, the polymer may be hydrolyzed upon reaction with water. The rate of degradation may be fast or slow, depending on the materials used to form the matrix, and in some cases, the rate of degradation may be controlling the composition of the material. For example, the material may comprise polylactic acid and/or polyglycolic acid, and the rate of degradation may be controlled by controlling the ratio of polylactic acid to polyglycolic acid within the material.

In one set of embodiments, the material defining the matrix may be chosen to be a low signaling material, or a material having low instructive capacity. As used herein, a “low signaling” material or a “low instructive capacity” material is a material that does not have the ability to signal cells to a large extent, for example, to induce certain behaviors (e.g., growth kinetics or differentiation behavior) and/or phenotypes in the cells, such that cells exposed to the low signaling material behave in substantially the same way in the presence and in the absence of the low signaling material when the cells are placed on a two-dimensional surface containing the low signaling material, or when the low signaling material is dissolved or suspended in the cell culture media. In a three-dimensional low signaling matrix, however, the three-dimensional matrix may induce cells contained within the matrix to behave in a certain way or to express a certain behavior and/or phenotype due to the three-dimensional structure of the matrix, rather than due to any inherent chemical reactivity or specific recognition of the material with respect to the cells.

Accordingly, a low-signaling matrix material can be readily identified by those of ordinary skill in the art by establishing three cell cultures: a first control cell culture (with no low signaling material present), a second cell culture where the cells are grown on a two-dimensional surface containing the low signaling material, and a third cell culture where the cells are contained within a three-dimensional matrix containing the low signaling material. In a low-signaling material, the cells of the first and second cell cultures will display substantially identical behaviors and/or phenotypes (i.e., they will not be statistically distinguishable), while the third cell culture will display a behavior and/or phenotype that is distinct from the first and second cell cultures (i.e., statistically distinguishable). For example, the third cell culture may display different differentiation or differentiation behavior than the other cell cultures.

A specific non-limiting example is described below in the examples using collagen I gel. In this example, the cells within the matrix undergo bone differentiation, but not cartilage differentiation. It is believed that this is because collagen I is the natural extracellular matrix present in skin and bone, which may instruct cells to differentiate into these lineages, preventing the differentiation into cartilage, where collagen II dominates. Thus, materials having low instructive capacity may allow spontaneous differentiation into cartilage and adipose tissue as well as bone tissue.

As previously mentioned, certain aspects of the invention are directed to culturing cells, such as fibroblasts or stem cells, within a three-dimensional matrix such as that described above. Virtually any type of cell can be cultured within the three-dimensional matrix, in conjunction with appropriate culture media and environmental conditions. Those of ordinary skill in the art will be familiar with cell cultures of various types, including suitable media and environmental conditions associated with such cell cultures. Any suitable cell type or types may be selected, along with appropriate culture media and conditions, and the cells may be incorporated within the three-dimensional matrix as described herein, and subjected to the appropriate environmental conditions.

For example, in one aspect, fibroblasts may be cultured within a three-dimensional matrix. In other aspects, hepatocytes, myocytes, chondrocytes, and/or osteocytes may be cultured within the three-dimensional matrix. In some instances, the three-dimensional matrix may include a co-culture, i.e., a culture comprising more than one type of cell at the start of the culture.

In one set of embodiments, the invention is directed to culturing fibroblasts under conditions such that at least some of the fibroblasts give rise to non-fibroblast multipotent cells or progenitor-like cells, i.e., cells that can give rise to several other related cell types. Thus, the fibroblasts may acquire distinct characteristics, such as protein expression levels, that are indicative of other types of cells other than fibroblastic cells, for instance, characteristics indicative of osteogenic differentiation. Such conditions can occur in vitro or in vivo. The fibroblasts may acquire such characteristics by culturing them within a three-dimensional matrix, such as those described herein.

For example, in one set of embodiments, the fibroblasts are induced to form osteoblast-like cells within a three-dimensional matrix. For instance, the fibroblasts can be exposed to osteogenic media, for example, comprising mesenchymal cell growth supplement, which can induce the fibroblasts to acquire characteristics of osteogenic cells. As mentioned, the mesenchymal cell growth supplement may contain, in some instances, various tissue-specific differentiation factors which enable cells to differentiate, although there may not necessarily be any osteogenesis-promoting growth factors present within the mesenchymal cell growth supplement.

Osteogenic cells (or tissues) can be identified using techniques known to those of ordinary skill in the art, for instance, Ca²⁺ mineralization assays (since osteogenic cells may form a mineralized matrix comprising Ca²⁺) such as von Kossa staining, OPN (osteopontin) assays (since osteogenic cells may exhibit intracellular and/or extracellular osteopontin), collage type I assays (since osteogenic cells may express collagen I), Runx2 transcription factor assays (since osteogenic cells may express Runx2), e.g., by Western blot or immunofluorescence detection, or alkaline phosphatase (ALP) assays (since osteogenic cells may express alkaline phosphatase activity), e.g., by an enzymatic activity determination using a chromogenic substrate. In some cases, such differentiation may occur even without the presence of specific growth factors, such as bone morphogenic proteins (BMPs), that are known to promote osteogenesis in fibroblastic cells. Non-limiting examples of suitable assays are described in the examples, below.

Without wishing to be bound by any theory, it is believed that, under certain conditions, fibroblasts may be induced to dedifferentiate to form osteogenic cells due to exposure of the fibroblasts to osteogenic media in the presence of a suitable three-dimensional environment (e.g., due to a three-dimensional matrix), which mimics conditions found in nature and thereby enhances the ability of the fibroblasts to dedifferentiate to form osteogenic cells. In particular, it is believed that fibroblasts can undergo osteogenic dedifferentiation due to the common mesenchymal origin with adipogenic and chondrogenic lineages. In addition, it is believed that fibroblasts are inhibited from dedifferentiating to stem cell-type phenotypes in the presence of the immune system; thus, partial or total inhibition or suppression of the immune system (e.g., locally), in some embodiments, may facilitate fibroblast dedifferentiation under such conditions.

Accordingly, in some embodiments, the fibroblasts can take on a stem cell-type phenotype or the phenotype of another progenitor-like cell, for example, when exposed to a suitable three-dimensional environment such as a three-dimensional matrix, optionally while suppressing immune responses, e.g., by culturing the fibroblasts in the presence of one or more anti-inflammatory factors, for instance, proteins such as interleukin 4 (IL4), interleukin 6 (IL6), interleikin 10 (IL10), transformin growth factor beta-1 (TGFβ-1), or extracellular adherent protein (Eap) that inhibits host leukosite recruitment; bioactive lipids such as prostaglandin E2 (PE2); glucocorticoids or corticosteroids such as prednisone; or non-steroidal anti-inflammatory drugs such as ibuprofen, naproxen, COX-2 inhibitors, etc. As mentioned above, such cultures can be performed in vivo or in vitro.

A fibroblasts taking on a stem cell-type phenotype, according to certain embodiments of the invention, may synthesize osteopontin perinuclearly, and eventually within the extracellular matrix, and may be accordingly determined. Osteopontin has been implicated in remodeling processes such as bone resorption, and angiogenesis, or wound healing. The osteopontin may be stored in granule-like structures. The fibroblasts, upon acquiring a stem cell-like phenotype, can also, in some cases, secrete a metalloproteinase (MMP), for example, MMP-2 and MMP-9, which are collagenases. Accordingly, the detection of expression levels of markers such as OPN and MMP can be used to identify when a fibroblast has acquired a stem cell-like phenotype. Such markers can be identified using standard techniques, such as gel electrophoresis.

Fibroblasts taking on a stem cell-type phenotype can also be induced to differentiate into various types of cells or tissues in various embodiments of the invention. In some cases, at least some of the fibroblasts may be able to form various tissue-specific cell types after exposure to an appropriate tissue-specific differentiation factor. For example, at least some of the fibroblasts may form tissue-specific cells such as skin cells, bone cells, cartilage cells, fat cells, muscle cells, or the like, and in some cases, the cells may form appropriate tissues. Thus, one way to demonstrate the multipotential capacity of a fibroblast with a progenitor cell-type phenotype is to expose the fibroblasts to different specific differentiating medium in vitro. As non-limiting examples for dermal differentiation, some minimum components would be: 2% FBS, FGF-2 and insulin; for bone differentiation: 10% FBS, hydrocortisone-21-hemisuccinate, beta-glycerophosphate, ascorbic acid; for cartilage differentiation: R³-IGF-1, FGF-2, insulin, transferring, and TGF-beta; for adipose tissue differentiation: 10% FBS, insulin, dexamethasone, 3-isobutil-1-methylxanthine, panthotenate; for muscle differentiation: 10% FBS, EGF, insulin, dexamethasone, L-glutamine; etc. In some cases the tissues may further implanted into a subject, as further described below, for instance, for wound healing or tissue regeneration.

In some embodiments, the fibroblasts are subsequently removed from the three-dimensional matrix. This may be achieved, for example, using cell removal techniques known to those of ordinary skill in the art, for example, trypsin and/or ethylenediaminetetraacetate (EDTA) exposure, and/or by disrupting the three-dimensional matrix mechanically, for example, with a pipette or with ultrasound, etc. The cells may then be resuspended, centrifuged, plated, or the like using standard cell-culture techniques known to those of ordinary skill in the art.

Of course, the invention is not limited to only culture of fibroblasts, but includes culturing other types of cells as well. Other cell types could include any adult or embryonic stem cells as well as any fetal, post-natal, juvenile and adult differentiated such as skin derived cells such as epidermal keratinocytes, dermal macrophages, melanocytes; follicle heart cells; subcutaneous gland cells; smooth and skeletic muscle cells; heart derived cells such as chardiomyocytes, or cardiac fibroblasts; lung derived cells, fat cells such as adipocytes; bone derived cells such as osteoblast, osteoclast and osteocytes; cartilage derived cells such as chondrocytes; endothelial cells; stroma bone marrow cells, bone marrow endothelial cells; blood derived cells such as lymphocytes, granulocytes, macrophages, dendritic cells; lymphoid gland derived cells; central and peripheric nerve system derived cells such as neural glia cells, neurons and nerve cells; internal organ derived cells such as liver hepatocytes, pancreatic cells, kidney cells, blader cells; digestive track derived cells such as oral cavity cells, teeth cells, trachea cells, stomach cells; intestine cells; sexual organ derived cells such as from ovary and testicles structures; eye derived cells such as cornea cells, crystalline cells, retina cells; etc.

For example, in one aspect, the cells cultured within the three-dimensional matrix are stem cells. The stem cells may be embryonic stem cells or adult stem cells, and the cells may be pluripotent, totipotent, or multipotent. Those of ordinary skill in the art will be aware of stem cells, and techniques for identifying and culturing stem cells in vitro, e.g., using appropriate stem cell culture media and environmental conditions. Non-limiting examples include chondrocyte culture and phenotype maintenance; adult liver progenitor cell culture and differentiation; neural progenitor cell isolation, culture and differentiation, aortic endothelial cell culture and phenotype maintenance (see e.g., J. Kisiday, et al., “Self-assembling peptide hydrogel fosters chondrocyte extracellular matrix production and cell division: Implications for cartilage tissue repair,” Proc. Natl. Acad. Sci. USA, 99: 9996-10001, 2002; C. Semino, et al., “Functional differentiation of hepatocyte-like spheroids structures from putative liver progenitor cells in three-dimensional peptide scaffolds,” Differentiation, 71: 262-270, 2003; C. Semino, et al., “Entrapment of migrating hippocampal neural cells in 3-D peptide nanofibers scaffolds,” Tissue Eng., 10: 643-655, 2004; or E. Genové E, et al., “The effect of functionalized self-assembling peptide scaffolds on human aortic endothelial cell function,” Biomaterials, 26: 3341-3351, 2005.

In one set of embodiments, the stem cells are cultured within a three-dimensional matrix such that at least some of the stem cells remain as stem cells. Without wishing to be bound by any theory, it is believed that, under certain conditions, stem cells may be propagated using a three-dimensional matrix such that at least some of the stem cells are able to retain their stem cell characteristics, i.e., the stem cells do not further differentiate. For instance, the stem cells within a three-dimensional matrix, such as a low signaling matrix, may divide to form cell masses or tissue masses, for instance, embryoid bodies, in which some of the cells retain stem cell characteristics. The cells may be able to divide such that a portion of the cells retain stem cell characteristics due to the presence of the three-dimensional matrix, which may enhance the ability of the cells to properly differentiate such that a portion of the cells within the differentiated cell mass properly do not differentiate, thereby retaining their characteristics as stem cells. It is believed that this scenario mimics conditions found in nature, in which tissues that form from stem cells (e.g., during development) will nonetheless contain some stem cells, e.g., for subsequent regeneration, repair, etc. of the tissue. Such stem cells can also be subsequently removed from the cell mass using known techniques for stem cell isolation and identified as stem cells, and in some cases, such stem cells can be re-introduced into a three-dimensional matrix and the process repeated. The cells can be removed from the three-dimensional matrix and/or from the cell mass, using cell removal techniques known to those of ordinary skill in the art, as previously described.

Additionally, in certain cases, the stem cells can be induced to divide repeatedly to form various differentiated tissues, for example, osteogenic tissue. In some cases, the differentiated tissue may also contain stem cells in some cases, which can be subsequently identified and/or removed from the tissue, and optionally re-introduced into a three-dimensional matrix.

Accordingly, one embodiment of the invention provides a process that allows stem cells to be cultured in vitro for indefinite amounts of time, such that stem cells can be recovered from the cell culture when desired. For example, stem cells may be cultured using three-dimensional matrices for at least two days, for at least five days, at least seven days, at least fourteen days, at least three weeks, or even at least four weeks or longer, such that at least some of the stem cells are able to retain stem cell characteristics, and can be recovered when desired.

In contrast, however, similar two-dimensional systems (e.g., stem cells cultured on a surface), which lack the proper environmental cues, do not allow proper cell differentiation to occur in the stem cells, such that cell or tissue masses formed in such two-dimensional systems from stem cells will not contain undifferentiated stem cells. See Example 4 for an illustration.

In one set of embodiments, the stem cells may be cultured within a three-dimensional matrix in culture media that is substantially free of stem cell promoting factors, such as leukemia inhibitory factor (LIF), which are usually added to two-dimensional stem cell cultures in order to maintain at least some of the stem cells within the cell culture as stem cells. Generally, a stem cell promoting factor is a factor that, when added to a stem cell, allows the stem cell to maintain its phenotype. For instance, fetal bovine serum (FBS) may be used for maintenance of the embryonic stem cell phenotype for embryonic stem cells. As mentioned, the stem cells may be able to divide such that a portion of the stem cells retain stem cell characteristics due to the presence of the three-dimensional matrix. It is believed that the three-dimensional matrix mimics conditions found in nature, thus promoting the development of cell or tissue masses, such as embryoid bodies, in which some of the cells retain stem cell characteristics, even without the presence of stem cell promoting factors.

The stem cells can be identified as stem cells after culture for extended periods of time according to certain embodiments of the invention, for example, using standard stem cell identification techniques, such as the detection of expression of Oct4. Oct4 is a marker commonly used to identify stem cells, and is a member of the Oct family of transcription factors that are involved in regulation of tissue- and cell-specific transcription and in transcription of housekeeping genes. Oct4 expression generally increases for undifferentiated stem cells, but decreases as the stem cells differentiate. The expression of Oct4 can be determined using techniques know to those of ordinary skill in the art, for example, using an antibody directed against Oct4, along with detection methods such as ELISA, Western blotting, immunoprecipitation, or immunohistochemical techniques, as known to those of ordinary skill in the art. In one embodiment, visual inspection under a fluorescent microscope may be used to identify Oct4 expression by transfecting the stem cells with GFP (green fluorescent protein) or similar markers under the transcriptional control of the Oct4 gene. In general, one or more molecular markers can be used to identify and characterize multipotent or pluripotent cells in vitro from different tissues type. As examples, cells can be considered adult or embryonic progenitor cells using markers including, but not limited to: alkaline phosphatase, c-kit, Rex-1, nestin, osteopontin, Alpha fetoprotein, Flk-1, A2B5, ABCG2, STRO-1, SSEA-1,3,4, Sca-1, CD133, CD34, GDF-3, noggin, Oct3/4, Nodal, Notch, Brachyury, Sox-17, Sox-1, Pax family, BMP family, etc. In one set of embodiments, a cell can be identified as being multipotent or pluripotent by it capacity to differentiate into several cell types after a differentiation assay in vitro. As mentioned, a cell may demonstrate a stem cell phenotype by its capacity to functionally regenerate in vivo in a specific tissue or organ.

Stem cells grown within a three-dimensional matrix can then be subsequently induced to differentiate. For example, a stem cell may be induced to differentiate by reducing the amount of LIF or FBS, and/or increasing the amount of certain differentiating factor or adding certain differentiating factor such as a particular growth factor, cytokine, small chemical entity, a component of the extracellular matrix, or some change in a physical biophysical parameter such as 3-dimensionality, matrix stiffness, internal media flow, oxygen concentration, mass transfer phenomena including nutrient supply and toxin elimination, etc, or acquiring a certain critical cell density promoting cell-cell interaction, migration and cell-cell instruction, or cellular self-organization. As a specific example, in one set of embodiments, the stem cells may be induced to form osteoblast-like cells, in some cases, while within the three-dimensional matrix. For example, the stem cells may be exposed to osteogenic media, for instance, comprising mesenchymal cell growth supplement, which may induce the stem cells to differentiate and acquire characteristics of osteogenic cells, as discussed below. The mesenchymal cell growth supplement can contain various tissue-specific differentiation factors which enable cells to differentiate, although there may not necessarily be any osteogenesis-promoting growth factors present within the mesenchymal cell growth supplement, and osteogenic differentiation may be partially assisted due to the presence of the three-dimensional matrix.

The systems and methods of the invention can be used in a wide variety of applications according to various aspects, such as wound healing, regeneration, drug discovery, cell biology, or the like.

For example, one set of embodiments of the invention are directed to systems and methods of assaying drugs, e.g., for drug screening. In some cases, a three-dimensional matrix may include cells, such as stem cells or fibroblasts, and a candidate drug can then be brought into contact within the matrix such that the drug interacts with the cells within the matrix, for example, by diffusing into the matrix in order to interact with the cells. After exposure to the drug, the cells within the matrix may be studied to determine the effects of the candidate drug on the cells. For instance, the drug may cause the cells to increase or decrease proliferation, or the drug may alter the ability of the cells to differentiate or dedifferentiate. Such effects may be studied, for example, in comparison to controls in which no drug is brought into contact with a three-dimensional matrix containing cells. Non-limiting examples of suitable techniques to study the cells include those described herein, as well as others known to those of ordinary skill in the art, for example, using protein assays (e.g., SDS-PAGE, gel electrophoresis, etc.) or the like. The use of a three-dimensional matrix as described herein may facilitate drug assays in some cases as the cells are present in an environment that may generally mimic in vivo conditions, as opposed to conventional two-dimensional assay techniques, in which the cells are plated onto the surface of a substrate.

In another set of embodiments, a three-dimensional matrix, optionally containing cells, may be applied to the wound (e.g., inserted or implanted into the wound) of a subject to facilitate wound healing. The term “patient” or “subject” as used herein includes mammals such as humans, as well as non-human mammals such as non-human primates, cows, horses, pigs, sheep, goats, dogs, cats, rabbits, or rodents such as mice or rats. The wound may be caused by physical trauma (e.g., a cut), or by a burn, such a chemical burn or a temperature burn. In some cases, the three-dimensional matrix applied to the wound of a subject is also covered in some fashion to protect the matrix, e.g., with a bandage, a membrane, a waterproof cover, etc.

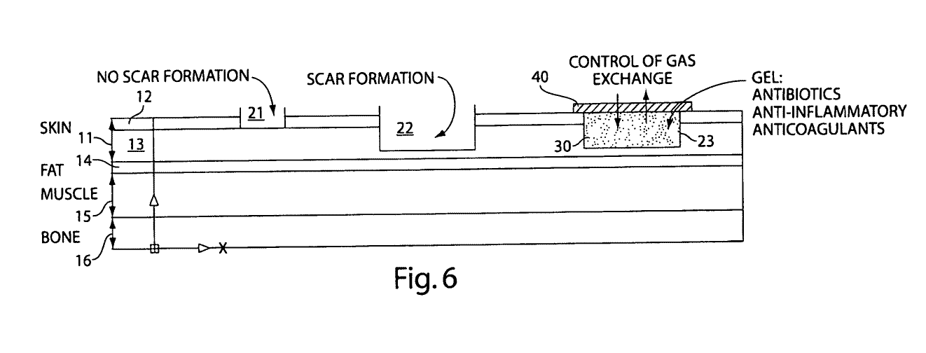

A non-limiting example of an embodiment of the invention is shown in FIG. 6. In this figure, a tissue 10 is shown comprises multiple layers: skin 11 (comprising dermis 12 and epidermis 13), fat layer 14, muscle layer 15, bone 16, etc. Although smaller wounds such as wound 21 may not result in scar formation, larger wounds such as wound 22 may result in scar formation. In wound 23, a three-dimensional matrix 30 in the form of a gel has been added. Optionally, the three-dimensional matrix may contain other factors, such as antibiotics, anti-inflammatory factors, anticoagulants, etc. Also shown in FIG. 6 is optional covering 40, which is used to protect the wound and/or to control gas exchange with wound 23, for example, to reduce the concentration of oxygen in the wound, as discussed below.

In some cases, the three-dimensional matrix may be removed from the wound of the subject after a suitable time. For instance, after some healing has occurred in the wound, the three-dimensional matrix may be removed, and in some cases, replaced with another three-dimensional matrix, or with a conventional bandage. In other cases, however, the three-dimensional matrix may become incorporated within the healing wound, for example, if the three-dimensional matrix includes biocompatible and/or contains biodegradable components. Thus, no additional step of removing the three-dimensional matrix is necessary.

The three-dimensional matrix may facilitate wound healing, for example, by providing an environment in which cells can grow and divide. Such an environment may encourage the growth of stem cell and/or progenitor-like cells (e.g., from fibroblasts, as previously discussed), which can facilitate wound healing. In some cases, the three-dimensional matrix may facilitate the transfer of nutrients, etc. to the cells, and in some embodiments, the three-dimensional matrix can also serve to prevent contaminants (e.g., foreign debris, bacteria, etc.) from entering the wound site, for example, if the three-dimensional matrix is covered in some fashion, and/or due to the size or porosity of the three-dimensional matrix.

In some embodiments, the three-dimensional matrix, when used for wound healing, may comprise cells. In other embodiments, however, the three-dimensional matrix is free of cells when the matrix is applied to the wound of a subject. In some cases, cells such as fibroblasts from the subject can then migrate from the wound into the three-dimensional matrix. The three-dimensional matrix may also contain factors, such as chemotaxis factors, to facilitate the migration of cells from the subject into the three-dimensional matrix in some instances.

Other factors may be present as well, for example, antibiotics, anticoagulants, anti-inflammatory factors, etc. As discussed, in some cases, cells such as fibroblasts, when located within the three-dimensional matrix in an environment in which the immune response is suppressed, may acquire other characteristics, such as a stem cell-type phenotype or the phenotype of another progenitor-like cell. Such cells may facilitate wound healing or tissue regeneration. For instance, the cells may be able to produce a substantial number of progenitor cells, and/or the cells may be able to differentiate to form multiple types of cells that are necessary to effect wound healing (e.g., muscle cells, bone cells, fat cells, connective tissue, etc.). It is believed that, in some cases, the environment surrounding the cells (i.e., the body) gives the necessary cues to cause differentiation to occur, and thus, the gel does not necessarily need any structural components in order to cause differentiation of the cells into the proper types of cells at their proper locations.

Thus, in yet another set of embodiments, the three-dimensional matrix may be used to regenerate tissue, for example, tissue that has been severed or lost from the body. For example, the tissue may have been severed by a surgical procedure, or as the result of an amputation, for example, a severed digit. In some cases, for example, for fairly large amputations or large wounds, a clamp or other mechanical device may be used to immobilize the three-dimensional matrix with respect to the underlying tissue.

If the three-dimensional matrix comprises cells when applied to the wound of the subject, such cells may arise from the subject (i.e., autologous cells), or arise from another subject. If the cells arise from the same subject, the cells may be taken from another location within the subject (which may be from the same or different species). For example, a tissue sample, such as biopsy, or a blood sample may be withdrawn from a subject, and cells such as fibroblasts isolated from the sample. The cells may then be introduced into the three-dimensional matrix, as described herein, and the three-dimensional matrix applied to a wound.

Thus, in one set of embodiments, a three-dimensional matrix is implanted within a subject, optionally containing cells such as fibroblasts. For example, the three-dimensional matrix may be implanted as a prosthetic device, for example, as a cartilage replacement, or the device may be implanted as a therapeutic device, e.g., a device which promotes wound healing or regeneration. The subject may be any suitable subject in need of such treatment.

In one set of embodiments, a tissue (e.g., a biopsy) is removed from the subject, fibroblasts (or other cells) extracted from the tissue, then the fibroblasts are added to a three-dimensional matrix, which is implanted back into the subject. In some cases, this procedure may occur very rapidly, e.g., on the order of a day or less (e.g., hours), such that the procedure can be completed while the subject is still in surgery. Thus, the cells are not cultured outside of the subject, but are merely extracted from the subject and then re-implanted back into the subject. In other embodiments, however, the cells may be cultured, and in some cases induced to form other types of tissues, prior to implantation back into the subject, or into another subject. In still another set of embodiments, tissues produced by the three-dimensional matrix may be implanted into a subject. For example, a tissue may be removed from a three-dimensional matrix, using removal techniques such as those previously described, and the tissue implanted into the subject.

In some embodiments of the invention, tissue regeneration and/or wound healing may be facilitated by reducing the concentration of oxygen in the wound or regeneration site, and/or by reducing blood flow to the wound or regeneration site. For instance, a device can be provided in certain embodiments that comprises a three-dimensional matrix (e.g., as previously described), an oxygen restricting component in fluidic communication (gaseous and/or liquid) with the three-dimensional matrix, and/or a blood flow restricting component in fluidic communication with the three-dimensional matrix and/or the oxygen restricting component.

In one embodiment, regeneration may be facilitated by reducing or limiting oxygen to the regeneration site, for instance, by using a semipermeable membrane. Oxygen may be restricted such that the oxygen concentration within the site is at a level of less than about 21%, for example, less than about 18%, less than about 15%, less than about 10%, or less than 5%. The oxygen may be restricted using a component that reduces access of air to the site, for example, a bandage, a cover, or a membrane. Examples of such membranes include, but are not limited to, semipermeable polyolefin (e.g., EXAIRE supplied by Tredegar Corporation, Richmond, Va., USA), expanded polytetrafluoroethylene (e.g., GORE-TEX supplied by W.L. Gore & Associates, Elkton, Md., USA), polyurethane foam (e.g., FLEXZAN supplied by Dow Hickam Pharmaceuticals, Sugar Land, Tex., USA), silicone and polytetrafluoroethylene (e.g., SILON-TSR, supplied by Bio Med Sciences, Allentown, Pa., USA), or the like.

In another embodiment, wound healing may be facilitated by reducing blood flow to the regeneration site. For example, prior to insertion or implantation of a three-dimensional matrix, arteries and/or veins within the wound site may be cauterized using routine procedures known to those of ordinary skill in the art. See, for example, U. Buchler, “Traumatic soft-tissue defects of the extremities. Implications and treatment guidelines,” Arch. Orthop. Trauma. Surg., 109:321-329, 1990, or S. V. Zachary and C. A. Peimer, “Soft-tissue management of complex upper extremety wounds. Salvaging the unsalvageable digit,” Hand Clinics, 13 (2): 239-249. Inhibition of blood flow may facilitate healing since the reduction of blood flow may reduce the immune response. Of course, typically, blood flow to the wound site is not completely cut off. In yet another embodiment, an anticoagulant may be used, e.g., to reduce the chance of blood clot formation.

In some embodiments, the matrix may comprise additional factors, which may be useful in promoting wound healing and/or regeneration. For example, the matrix may comprise various hormones, antibodies, enzymes, drugs or other pharmacological agents, or other factors, such as cell signaling factors, antibiotics, etc. These factor may include anti-inflammatory factors, for instance, proteins such as interleukin 4 (IL4), interleukin 6 (IL6), interleukin 10 (IL10), transformin growth factor beta-1 (TGFβ-1), the extracellular adherent protein (Eap) that inhibits host leukosite recruitment; bioactive lipids such as prostaglandin E2 (PE2); glucocorticoids or corticosteroids such as prednisone; non-steroidal anti-inflammatory drugs such as ibuprofen, naproxen, COX-2 inhibitors, etc. Further non-limiting examples include growth factors such as human recombinant EGF (hrEGF), human recombinant basic FGF (hrFGF-2); hormones such as insulin; antibiotics such as penicillin, gentamicin, amoxillin, etc.

The cells of the invention may be treated with therapeutics or other compounds before or after they are seeded in the matrix. For instance the fibroblasts, and progeny thereof, can be genetically altered. Genetic alteration of cells includes all transient and stable changes of the cellular genetic material which are created by the addition of exogenous genetic material. Examples of genetic alterations include any gene therapy procedure, such as introduction of a functional gene to replace a mutated or nonexpressed gene, introduction of a vector that encodes a dominant negative gene product, introduction of a vector engineered to express a ribozyme and introduction of a gene that encodes a therapeutic gene product. Natural genetic changes such as the spontaneous rearrangement of a T cell receptor gene without the introduction of any agents are not included in this concept. Exogenous genetic material includes nucleic acids or oligonucleotides, either natural or synthetic, that are introduced into the dermal mesenchymal stem cells. The exogenous genetic material may be a copy of that which is naturally present in the cells, or it may not be naturally found in the cells. It typically is at least a portion of a naturally occurring gene which has been placed under operable control of a promoter in a vector construct. Methods for delivering nucleic acids to cells and expressing them are well known in the art.

### EXAMPLES

Introduction: In the following examples, murine embryonic stem cells (mESC) and primary mouse embryonic fibroblasts (MEF) were chosen as cell sources in order to assess their differentiation capacity into osteoblast-like cells in a 3D-culture system. A transgenic derivative of the mouse embryonic stem cell line R1 expressing the green fluorescent protein (GFP) under the transcriptional control of the Oct4 gene, and primary MEF (strain C57BL/6) in a three-dimensional culture technique that uses a synthetic nanofiber scaffold as extracellular matrix analog were used. Parallel experiments were also carried out on classical culture dishes (2D) in order to compare the differentiation capacity between both culture systems under osteogenic conditions. These examples demonstrate that both 2D- and 3D-culture systems promoted differentiation of mESC into cells with osteoblast-like phenotype expressing bone markers including osteopontin (OPN), collagen type I (Coll I), alkaline phosphatase (ALP) and calcium mineralization (CM). Interestingly, differentiation of MEF into osteoblast-like cells appeared in the 3D-culture system, but not the 2D-culture system. The differentiated cells in this case also presented similar osteoblastlike phenotype expressing ALP, Coll I, Runx2 transcription factor, and CM. Furthermore, MEF cultured in the 3D-system with regular growth medium (control medium) for one to two weeks prior to osteogenic differentiation expressed OPN, presented high mitotic capacity, and up-regulated several active metalloproteinases, suggesting that the 3D nanoscaffold system promoted MEF to turn into a “progenitor-like intermediate” with osteogenic potential. These examples thus show that the 3D-culture system can be used to explore the potential of this progenitor-like intermediate to differentiate into other mesenchymal tissues including cartilage, muscle, and fat.

### Example 1

### Materials and Methods

Certain protocols and methods that are useful in various embodiments of the invention are now described.

Cell culture. Mouse Embryonic Fibroblasts isolated from C57BL/6 embryos at day 14 were obtained from the ATCC and expanded in fibroblast medium (FM), which contains DMEM high glucose with 15% (v/v) FBS, 4 mM L-glutamine, 100 U/ml penicillin and 0.1 mg/ml streptomycin. Cells were cultured in various 3D and 2D environments to observe cell behavior and differentiation. One three-dimensional culture technique used is a self assembling peptide structure. This technique included the encapsulation of cells into RAD16-I peptide (BD™ PuraMatrix™ Peptide Hydrogel, BD Biosciences). This peptide had the sequence AcN-RADARADARADARADA-CONH₂ (SEQ ID NO: 1).

Briefly, the procedure was as follows. A suspension of cells in 10% sucrose was mixed with an equal volume of liquid RAD16-1 peptide solution (0.5% w/v, pH 3.5 in 10% sucrose) at a final concentration of 2×10⁶ cells/mL, 0.5% of RAD16-I in 10% sucrose. Then, the cell-peptide suspension was loaded into transwell inserts (100 microliters/insert; 10 mm diameter, 0.78 cm² area, pore size of 0.2 micrometers, Nalge Nunc International, IL) and immediately equilibrated with 200 microliters of ESCM (embryonic stem cell medium) without LIF (leukemia inhibitory factor), added at the bottom of inserts, to initiate peptide gel formation by a self-assembling process.

In other experiments with RAD16-I, MEF cells (<8th passage) were trypsinized from 75 cm2 culture flasks, suspended in sucrose 20% and mixed with the self-assembling peptide RAD16-I (BD, Puramatrix) 0.5% to obtain a final MEF concentration of 2·10⁶ cells/ml and 0.25% of RAD16-I. This suspension was seeded into inserts (Millipore) and jellified by addition of FM. After jellification, FM was added on the hydrogel. Incubation was performed at 37° C. with 5% CO2 and medium was changed every 2 days.

The RAD16-I peptide is an amphiphilic molecule which can adopt a beta (β) sheet configuration in water containing a hydrophobic face (e.g., the —CH₃ groups of the alanine residues) and a hydrophilic face (e.g., the —COOH groups of the aspartic acids and the —NH₂ groups of the arginine groups). When the anion strength is increased or pH values raised to neutrality (i.e. physiological salt concentrations, culture media, buffers, etc.) the peptides may spontaneously self-assemble in an anti-parallel arrangement, forming a network of interweaving fibers which can be several micrometers in length, with an average thickness of about 10 nm, and pores of about 5 nm to about 200 nm in diameter. For purposes of illustration, an electron microscopy picture of a nanofiber network of a self-assembling peptide scaffold is shown in FIG. 1C (the bar is 250 micrometers). During the self-assembling process a nanofiber network may develop around the cells, encapsulating them in a 3 dimensional (“31)”) environment.

After formation, culture medium was added at the top of the formed hydrogel and changed three times during the first 60 min. Thereafter, peptide scaffold cultures were maintained at 37° C. in a humidified incubator equilibrated with 5% CO₂.

3D gels were also produced using Agarose and collagen. For the experiments with Agarose, the same cells were suspended in FM. This cell suspension was mixed with an equal volume of 0.5% agarose in sucrose 10% to obtain a final cell density of 2·10⁶ cells/ml in 0.25% agarose. The cell suspension was briefly cooled until jellification of the agarose. Gels were equilibrated in FM and incubated as described above.

For the experiments with collagen-I, the same cells were suspended in FM. This cell suspension was mixed with an equal volume of previously neutralized collagen-I (BD) to obtain a final cell density of 2·10⁶ cells/ml in 0.2% collagen. The suspension was put in inserts and let jellify at 37° C. Gels were cultured in FM and incubated as described above.

Osteogenic differentiation of mouse embryonic stem cells (mESC). Mouse embryonic stem cell line R1, which is transgenic for green fluorescent protein (GFP) expression under the control of the Oct-4 promoter (ES R1 Oct4-GFP cells), were used in various examples, as described below.

The ES R1 Oct4-GFP cells were maintained at 37° C. in humidified air with 5% CO₂ in ES cell medium. The ES cell medium was Dulbecco's Modified Eagle's Medium (DMEM, 4500 mg/mL glucose, GIBCO) containing 1000 U/mL recombinant mouse leukemia inhibitory factor (Chemicon International) to maintain the ES cell pluripotent characteristics, 15% FBS (fetal bovine serum, Hyclone, UT), 1 mM sodium pyruvate (GIBCO), 0.1 mM non-essential amino acids (GIBCO), 4 mM L-glutamine (GIBCO), 1% (v/v) penicillin-streptomycin (GIBCO), and 0.1 mM beta-mercaptoethanol (β-mercaptoethanol, Sigma).

ES cell differentiation was induced using standard methods, as follows. Briefly, ES cells were cultured in suspension at 1.5×10⁵ cells/mL in 10 mL of ESCM without LIF in 10 cm non-adherent bacteriological Petri dishes (VWR) for 8 days, when the embryoid bodies (EB) formed. The EB cultures were maintained at 37° C. in humidified air with 5% CO₂ and fed every 3 days by allowing the EB to settle in a tube, replacing medium, and gently pipetting with a wide-bore pipet into fresh Petri dishes.

Fluorescence microscopy was used to monitor GFP expression during EB formation. The EB were harvested and allowed to settle in a tube. The medium was removed and EB were gently treated with trypsin-EDTA 1× (GIBCO). The resultant EB-derived cells were encapsulated into the peptide scaffold (3D cultures) at a final concentration of 2×10⁶ cells/mL, as described above, and cultured in control medium (ES medium in the absence of LIF). 2D cultures, in regular culture plates, were also performed as controls. The EB-derived cells were plated into 12-well plate containing control medium (ES medium in the absence of LIF) at a concentration of 760,000 cells/mL (200,000 cells/cm²). The differences in the number of cells seeded between 2D and 3D cultures was used to maintain more similar cell densities between the systems: cells at 2×10⁶ cells/mL in the 3D culture presented a similar cell-to-cell distance as the 2×10⁵ cells in the 2D culture.

Cells, both from the 2D and 3D cultures, were allowed to grow in mESCM without LIF for 2-8 days before osteogenic induction (FIG. 1A). Then, the control medium was changed to osteogenic medium, which was Dulbecco's Modified Eagle's Medium (DMEM, 4500 mg/mL glucose, GIBCO) containing the Osteogenic SingleQuot kit (Cambrex), which included mesenchymal cell growth supplement (“MCGS”), 1% (v/v) penicillin-streptomycin, 4 mM L-glutamine, 0.05 mM ascorbate, 10 mM beta-glycerophosphate (β-glycerophosphate), and 0.1 micromolar dexamethasone. Additionally, 50 nM of 1-alpha,25-(OH)₂ vitamin D₃ (1α,25-(OH)₂ vitamin D, Sigma) was added to the osteogenic medium. The medium was changed every 2 days. The cultures were maintained for 8-22 days in osteogenic medium at 37° C. in humidified air with 5% CO₂.

Osteogenic differentiation of Mouse Embryonic Fibroblasts (MEF). Mouse embryonic fibroblasts (MEFs) were purchased from ATCC(SCRC-1008). They were isolated from mouse embryos derived from C57BL/6 mothers at 14 days of gestation (E14). The MEFs were expanded prior to osteogenic induction in MEF culture medium of DMEM (high glucose) containing 10% FBS (fetal bovine serum, Hyclone), 4 mM L-glutamine (GIBCO), and 1% (v/v) penicillin-streptomycin (GIBCO). The differentiation experiments were carried out using cells between passages 3 and 5. The MEFs were encapsulated into the peptide scaffold (3D cultures) at a final concentration of 2×10⁶ cells/mL, as described above (3D cultures). 2D cultures were also performed by culturing MEFs into 12-well regular culture plates. Both the 2D and 3D cultures were maintained in MEF culture medium for 2-4 days (FIG. 1B). Next, the MEF medium was changed by osteogenic medium (same as in mESC osteogenic induction section). Also, 10 mM of 1-alpha,25-(OH)₂ vitamin D₃ (Sigma) was added to the osteogenic medium as previously described for mouse-derived NIH 3T3 fibroblasts. The cultures were maintained for different time periods (FIG. 1B, STAGE 3) in osteogenic medium at 37° C. in humidified air with 5% CO₂. The medium was regularly exchanged with fresh osteogenic medium.

Additional differentiation assays: MEFs were cultured in the 3D hydrogels with FM for 12 days and after that, medium was changed to differentiation medium. MEFs were cultured in differentiation medium for 21 days. As Controls, MEFs were cultured for 33 days in regular fibroblast medium. For osteogenic differentiation, the medium was DMEM high glucose with 10% FBS, 1% (v/v) Penicillin streptomycin, 4 mM L-Glutamine, 0.05 mM ascorbate, 10 mM α-glycerophosphate, 0.1 μM dexamethasone and 20 nM 1γ,25-(OH)₂ vitamin D3. For the chondrogenic differentiation, the medium was provided by Cambrex (CC-4408). For the adipogenic differentiation, the medium was DMEM with 10% FBS, 8 μg/ml biotin, 4 μg/ml pantothenate, 0.5 mM 3-isobutyl-1-methylxanthine, 1 μM dexamethasone and 10 μg/ml insulin.

Phenotype assignment. Assignment of osteoblastic, chondrogenic or adipogenic phenotype was performed on MEFs after a differentiation assay by Von Kossa, Toluidine blue and Nile Red staining, respectively. Stainings were performed as described below.

Cell isolation from peptide scaffold 3D cultures. Cells from the peptide scaffold cultures (3D cultures) were harvested by treatment with trypsin-EDTA (0.05% trypsin, 0.53 mM EDTA-4Na in HBBS (Hank's balanced salt solution), without calcium or magnesium, GibcoBRL), and by disrupting mechanically with a micropipette until single cells were obtained as observed by phase microscopy. The released cells were washed with complete culture medium, counted, resuspended as needed, and subsequently seeded on regular culture dishes. These isolated cells were cultured on regular culture dishes and analyzed by immunofluorescence (for a variety of cellular markers such as osteopontin, Oct4, etc.) or used to perform cell kinetics experiments.

Von Kossa staining for mineralization. Mineralized aggregated cells were identified by the von Kossa method for mineralized calcium on days 17-20 of osteogenic induction. Briefly, 2D and 3D cultures were washed twice with 10% PBS (phosphate buffered saline, 1 mM KH₂PO₄, 137 mM NaCl, 2.7 mM KCl, pH 7.4, Roche Diagnostics Corp.) and fixed with 1% PFA (p-formaldehyde, J. T. Baker) in PBS for 1 hour. Then, the cultures were strictly rinsed with deionized water until the PFA in PBS solution was completely removed. In the 3D cultures, a few drops of 2% agarose solution were added on the top and left at RT (room temperature, about 25° C.) until gelification, in order to more easily manipulate the samples during the following steps. A volume of 5% (w/v) silver nitrate (AgNO₃, Sigma) solution in water, enough to cover the cell cultures was added, and left to react in the dark for 1 hour. The cultures were then washed with deionized water in order to remove the excess of AgNO₃ solution. The calcium mineralized nodules stained black and were analyzed by visual inspection. Toluidine blue and Nile Red staining were performed as previously described. See i.e., Tchoukalova Y D, Harteneck D A, Karwoski R A, Tarara J, Jensen M D (2003) J Lipid Res 44:1795-1801 and Geyer G, Linss W (1978) Acta Histochemica 61:127-134.

Immunofluorescence analysis. Immunostaining was used to detect the presence of osteopontin, a non-collagenous protein present in natural bone matrix and considered as an early osteogenic marker, in the isolated cells from 2D and 3D cultures after the osteogenic induction of ESC and MEFs. Osteopontin was also assayed on isolated MEFs after being cultured into the peptide scaffold and maintained in MEF culture medium for 15 days and on MEF 2D cultures maintained in MEF culture medium for the same time period in order to see the influence of the 3D culture system on osteopontin expression prior to osteogenic induction. In addition, Oct4 was assayed on ES cell and MEF cultures before and after the differentiation process. Isolation of cells after osteogenic induction either from 2D or 3D cultures was necessary in order to avoid interferences from mineralized calcium in the detection of above mentioned markers.

The cells from 2D cultures were harvested with 0.05% trypsin. The released cells were washed with complete culture medium, counted, resuspended as needed, seeded on regular culture dishes and allowed to grow for 4-6 days in their culture medium before assaying them. Cells from 3D cultures were isolated as described above with respect to cell isolation from peptide scaffold 3D cultures. Isolated cells obtained as explained above were cultured in multi-well plates, fixed with 1% PFA in PBS for 1 h, washed twice with PBS, and incubated with blocking buffer which was composed of 2% (v/v) FBS, 0.1% (v/v) Triton X-100 (Sigma), and 1% (v/v) DMSO (dimethylsulfoxide, Sigma) in PBS for 4 h at RT in an orbit shaker. Then, the cells were incubated overnight with the primary antibody at a concentration of 1 microgram/ml, washed three times with blocking buffer and incubated with the secondary antibody at a concentration of 1 microgram/ml for 2 h. The secondary antibodies were removed by washing three times with blocking buffer for detection under a Nikon microscope TE300. Antibodies specific for osteopontin (mouse monoclonal IgG, 200 microgram/ml), Oct4 (Oct4 (H-134) rabbit polyclonal IgG, 200 microgram/ml) and the secondary antibodies anti-mouse IgG-R (rhodamine conjugated, 200 microgram/0.5 ml), donkey anti-rabbit IgG-R (rhodamine conjugated, 200 microgram/0.5 ml), respectively, were each obtained from Santa Cruz Biotechnology.

Brachyury immunostaining. MEFs cultured in RAD16-I during 7, 11 and 15 days with FM were fixed and immunostained for Brachyury, using the following antibodies: goat anti-Brachyury (Santa Cruz, sc-17743) and donkey anti-goat HRP conjugated (Santa Cruz, sc-2020). Reaction with DAB substrate (Roche) showed the localization of Brachyury expression inside the cell mass.

In situ hybridization of Brachyury. MEF cultures in RAD16-I were fixed at days 7, 11 and 15. In situ hybridization was performed whole mount and onto 14 μm slices of the tissue-like cell mass obtained after culturing. Slices were obtained by cryosectioning of the cell-mass with Leica CM 3050 S cryostat using OCT compound as freezing support. The DNA probe used for the in situ hybridization was synthesized with the PCR DIG Probe synthesis kit (Roche 1636090) using primers kindly provided by D. Shaywitz (forward primer: CATGTACTCTTTCTTGCTGG (SEQ ID NO. 4); reverse primer: GGTCTCGGGAAAGCAGTGGC) (SEQ ID NO. 5). The in situ hybridization was performed following the company's instructions for embryos and for tissue sections. After hybridization of the DIG labeled probe, samples were immunostained for digoxigenin using sheep anti-DIG antibody (Roche 1636090910) and Donkey anti-sheep HRP-conjugated antibody (sc 2473). Reaction with DAB substrate (Roche) showed the localization of Brachyury mRNA.

Quantitative RT-PCR. mRNA was isolated with Lyzol (Invitrogen) from MEFs, cultured in RAD16-I and cultured in 2 dimensions (controls), at days 3, 7, 11 and 15. After degradation of cellular DNA, mRNA was retrotranscribed with Taqman Reverse Transcriptase. (Applied Biosystems, N808-0234) and quantitative RT-PCR was performed with this cDNA. SYBR green PCR kit (Qiagen 1718906) and commercial primers (Qiagen QT00163765) were used to amplify Sox9. Relative gene fold variations were determined by the 2^{-ΔΔCt} method using the ribosomal unit 18S as housekeeping gene.

RTPCR. mRNA was isolated with Lyzol (Invitrogen) from MEFs, cultured in RAD16-I and cultured in 2 dimensions (controls), at days 3, 7, 11 and 15. After degradation of cellular DNA, mRNA was retrotranscribed with Omniscript® (Qiagen, 205111). The cDNA obtained was amplified by PCR using Accuprime Pfx (Invitrogen, 12344-024). The primers were SEQ ID NO 4 and 5 as described above.

Alkaline phosphatase activity. Alkaline phosphatase enzyme activity (ALP) of 2D and 3D cultures was measured in triplicate cultures before and after the osteogenic differentiation. The cultures were rinsed twice with Tyrode's Balanced salt solution (TBS, 50 mM Tris base, 0.15 M NaCl, pH 7.4) and then the cells were incubated with 5 mM p-nitrophenyl phosphate (p-nitrophenyl phosphate disodium salt tablets, Sigma) solution in glycine buffer (50 mM glycine, 1 mM MgCl₂, pH 10.5) for 30 min at 37° C. ALP enzyme activity was calculated after measuring the absorbance of the reaction product formed, p-nitrophenol, at 405 nm on a spectrophotometer. Standards were prepared by consecutively diluting 10 mM p-nitrophenol (Sigma) solution in glycine buffer at concentrations ranging from 0.0001 mM to 0.06 mM. Total protein levels were determined using a protein detection kit (Bio-Rad) based on the method of Lowry in order to normalize total protein levels. The final APL activity was expressed in units/mg of protein (one unit will hydrolyze 1 micromoles of p-nitrophenyl phosphate per minute at pH 10.5).

Glycosaminoglycan quantification. GAGs were quantified using DMMB (1,9-dimethyl-dimethyleneblue). The samples were treated overnight with Pronase at 60° C. and centrifuged at 14000 g. The supernatant (40 μl) was incubated with 360 ml of a solution of DMMB (0.16% DMMB in 0.2% acid formic with 2.5 mg/ml sodium formate; pH 3.5) and read in a spectrophotometer with visible light (λ=535 nm).²⁷ Standards of chondroitin sulfate were used to prepare a calibration curve with values between 0 and 100 μg/ml.

Western blot analysis of collagen type I and Runx2. Collagen-II and Runx2 expression was analyzed in MEFs after chondrogenic and osteogenic differentiation protocols respectively. Agarose, collagen-I and RAD16-I were used as 3D-environments for MEFs; 2D cultures were used as control. Cells from 2D and 3D cultures were washed twice with PBS, scraped and suspended in lysis buffer which was composed of 0.1% (v/v) Triton X-100 (Sigma) and protease inhibitor cocktail (complete mini protease inhibitors cocktail, Roche) in PBS. The cell suspension was sonicated for 5 min (Aquasonic, model 50T, VWR) to complete cell disruption and centrifugated at 14,000 rpm for 5 min. Total protein levels at the supernatant fraction were determined using a protein detection kit (500-0116, Bio-Rad) based on the method of Lowry in order to normalize total protein levels. Protein loading buffer 4X (Invitrogen) containing SDS and beta-mercaptoethanol (P-mercaptoethanol) was added to the cell extract and heated at 80° C. for 10 min. Samples were loaded in a 10% PAGE (polyacrylamide gel electrophoresis) system (Invitrogen) equilibrated with MOPS SDS (morpholinopropane sulphonic acid sodium dodecyl sulfate) running buffer 20X (Invitrogen). After the run, the protein gel was transferred to a PVDF (polyvinylidene fluoride) membrane (Invitrogen) for 2 h using the transfer buffer (3.03 g Tris-basic, 14.4 g glycine, 200 mL methanol until 1 liter with deionized water). The PVDF membrane was washed before use, one time with methanol and after that, two times with transfer buffer. After the transfer, the membrane was incubated at RT for 2 h in an orbital shaker with blocking buffer, composed of 4% (w/v) non-fat powered milk, 0.1% (v/v) Triton X-100 in PBS. Then, the following primary antibodies were resuspended in the same blocking buffer and incubated with the membrane for 1 hour: anti-collagen-I NCL-COLL Ip rabbit polyclonal (1 mL, Novocastra), mouse anti-Collagen II antibody (NeoMarkers, MS-235-P1), dilution of 1:200, or anti-Runx2 (PEBP2αA) rabbit polyclonal antibody (Santa Cruz Biotechnology) at 1 microgram/mL. The primary antibody was removed by washing the membrane with blocking buffer three times for 30 min each time. The secondary antibody goat anti-rabbit IgG-HRP-conjugated, donkey anti-mouse antibody HRP conjugated (Santa Cruz, sc-2096), rabbit anti-PEBP2αA antibody (Santa Cruz, sc-10758) or donkey anti-rabbit antibody HRP conjugated (Santa Cruz, sc-2317) (Santa Cruz Biotechnology) was added at a concentration of 1 microgram/ml in blocking buffer and left to react with the primary antibody for 1 hour. The membrane was then rinsed 3 times for 30 min each time with blocking buffer in order to remove the excess of secondary antibody. The membrane was revealed using a chemiluminescent substrate reaction kit (Luminol Reagent, Santa Cruz Biotechnology). SeeBlue® Plus2 molecular weight standard (Invitrogen) was used to identify the molecular weight. Collagen-I from rats tail and mouse bone preparation were used as standard.

Nestin expression. MEFs were cultured with FM in 2D plates and in the different 3D hydrogels: agarose, Collagen-I, and RAD16-I. Gels were disrupted mechanically after 0, 5 and 10 days and seeded on regular 12 well plates. After 12 hours of incubation, cells were fixed with 4% paraformaldehyde. Immunostaining was performed using goat anti-nestin (Santa Cruz, sc-21248) as primary antibody, and chicken anti-goat FITC-conjugated (Santa Cruz, sc-2988) as secondary antibody. Percentage of FITC positive cells was counted as a measure of nestin expression.

Cell Cycle Arrest Assays with Staurosporine. Cultures of MEFs were incubated in FM with Staurosporine (20 nM) during 7 days while controls were incubated in FM. At day 6, a pulse of BrdU was applied overnight. MEFs were fixed at day 7 and stained with a mouse monoclonal antibody IGgl anti-BrdU FITC-conjugated (BD PharMingen, 33284×). To assess the dose-response effect of staurosporine on MEFs, these were incubated with different concentrations of staurosporine (0, 2, 20 nM) during 14 days. Visual inspection of the cell mass contraction along these 14 days was performed.

Kinetic growth rates determination. mES cells and MEFs isolated either from 2D or 3D cultures, control and osteogenic, as well as parental mESC and EB-derived cells were seeded in a 24-well plate at a known initial cell density in the appropriate culture medium. One 24-well plate was used for each cell type. The culture medium employed for differentiated ESC, including control and osteogenic experiments, and for EB-derived cells, was ESC medium without LIF. ESC medium was employed for parental mESC line. MEF medium was used for MEFs isolated from control and osteogenic experiments for both 2D and 3D cultures and also for parental MEFs line. The next day after the seeding, three wells were harvested and cells counted. This first value was taken as 0 h initial point of the growth curve. Every other day three wells were harvested and cells counted. Standard deviations were calculated for each point.

Zymography. Protease activity was determined through zymography using gelatin substrate casting gels adapted from published methods. Cells from 2D and 3D systems were isolated at a certain time point (after 20 days of culture into the gel in the case of 3D system), washed with serum-free medium, counted and dissolved in loading buffer in order to assay them for metalloproteinases. In addition, supernatants from MEF 2D and 3D cultures were collected at the same time point as above and diluted in loading buffer. Samples were normalized by loading the same number of cells per well. Those samples were subjected to electrophoresis with copolymerized 10% sodium dodecyl sulfate-polyacrylamide gels with 1 mg/ml gelatin. The gel was rinsed in Triton X-100 wash solution (2.5% in distilled water) for 2 hours with 3 changes of solution. It was then incubated in proteolysis buffer (50 mM Tris OH, 0.5 M NaCl, 50 mM CaCl₂, pH 7.8) overnight at 37° C. Gel staining was performed by using Coomassie blue staining (0.1% Coomassie blue, 5:5:2 distilled water, methanol, acetic acid) for 1 hour and distained in distaining solution for Coomassie blue (10% methanol, 82.5% H₂O, and 7.5% acetic acid).

### Example 2

This example illustrates osteogenic differentiation of mouse embryonic stem cells in 3D culture system, using techniques such as those described in Example 1. FIG. 1 illustrates a flow diagram of the differentiation protocols used in these examples. FIG. 1A is a schematic representation of the protocol used for the osteogenic induction of mouse embryonic stem cell line R1 Oct4-GFP. FIG. 1B illustrates the protocol for mouse embryonic fibroblasts, MEF. FIG. 1C is a scanning electron microscopy photograph of the self-assembling peptide nanofiber scaffold RAD16-I (PuraMatrix). The white bar is 250 nm.

In this example, the transgenic cell line ES R1 Oct4-GFP was used to obtain an embryonic cell lineage with osteogenic potential (mesoderm) by producing embryoid bodies (EB) following a classical differentiation protocol (FIG. 1A). Briefly, the embryonic cells were culture in mouse embryonic stem cell medium (mESCM) without leukemia inhibitory factor (LIF) on non-adherent Petri dishes to promote cell aggregation and EB formation (FIG. 1A, Stage 2). The EB presented reduced GFP expression indicating the loss of the pluripotent embryonic stem cell phenotype and the subsequent differentiation of embryonic tissues including ectoderm, mesoderm, and endoderm.

At this point, EB were collected and gently dissociated by enzymatic treatment with trypsin (FIG. 1A, Stage 3). The dissociated cells, called EB-derived cells (EB-dc), were cultured on classical culture dishes (2-dimensional system, 2D) or encapsulated in a synthetic peptide scaffold (3-dimensional system, 3D) (for illustration of the scaffold nanofiber structure, see FIG. 1C), and subsequently maintained in mESCM without LIF (mESCM/-LIF) for several days (FIG. 1A, STAGE 4). The culture medium was then replaced by osteogenic medium and maintained under these conditions for different periods of time (FIG. 1A, Stage 5). In addition, nondisaggregated EB were plated on culture dishes and cultured directly in osteogenic conditions, as previously described.

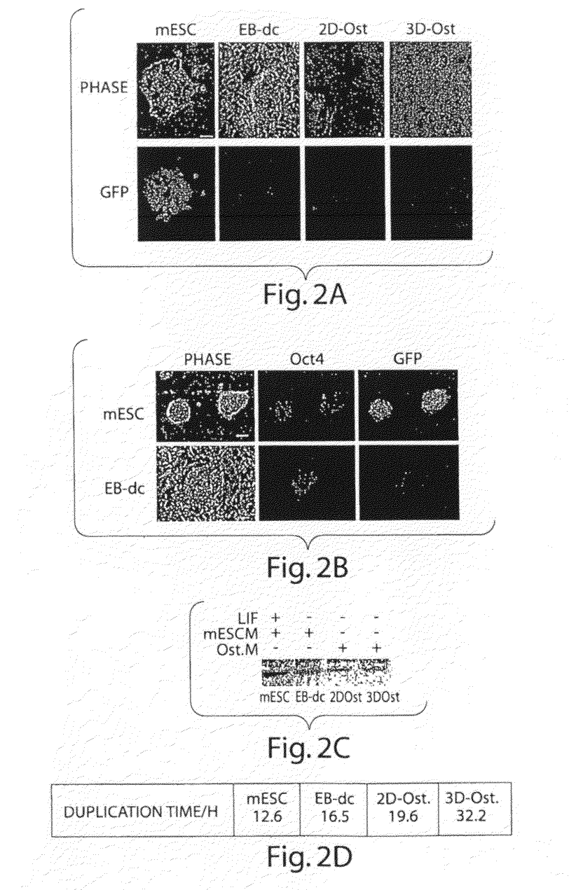

Oct4 expression was monitored at different stages of the process by visual inspection under fluorescent microscope (GFP expression), resulting in a progressive decrease of its expression to almost no detection at the end of the differentiation protocol (FIG. 2A). In order to confirm the fluorescent signal (by GFP) from ESC and EB-dc with Oct4 expression, the colonies from these stages were immunostained with an anti-Oct4 antibody, resulting in co-localization of GFP with Oct4 (FIG. 2B). In addition, western blot analysis was performed to follow the Oct4 expression during the entire differentiation process (FIG. 2C), reconfirming the previous GFP signal observed in FIG. 2A.

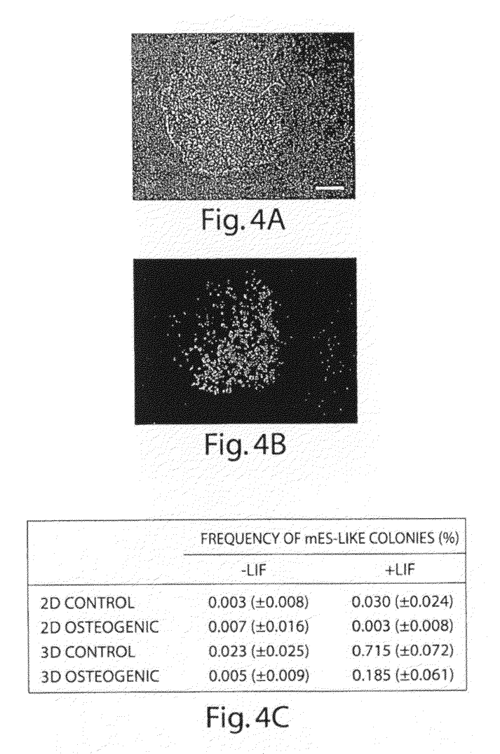

Specifically, FIG. 2A shows GFP expression of mESC during the differentiation process. mESC initially showed strong GFP expression, indicating the expression of the marker for pluripotentiality, Oct4, whereas after EB formation, GFP expression of EB-derived cells (EB-dc) dramatically decreased. At the end of the osteogenic induction, cells from both 2D and 3D cultures (2D-Ost and 3D-Ost) did not show any GFP expression, suggesting that cells were fully differentiated. The scale bar is 50 micrometers. FIG. 2B illustrates the Oct4 transcription factor immunofluorescence of mESC and EB-dc. The expression of Oct4 was in agreement with the GFP expression in observed in FIG. 2A. mESC showed an intense Oct4 expression which highly decreased after EB formation (EB-dc), although EB-dc cultures still presented a remaining population of cells that expressing Oct4. The scale bar is 50 micrometers. FIG. 2C shows a western blot analysis of the Oct4 transcription factor during the differentiation process: expression of Oct-4 by mESC; EB-dc; cells harvested from 2D-cultures after the osteogenic differentiation (2D-Ost); and cells harvested from 3D cultures after the osteogenic differentiation (3D-Ost). Oct4 expression decreased after EB formation and it was not detected after the osteogenic induction. FIG. 4D shows duplication times of cells (mESC, EB-dc, 2D-Ost, and 3D-Ost) during the differentiation process. Values were calculated from the proliferation curves of cells.

Next, the appropriate time period for the osteogenic differentiation was established by screening two different time points. The EB-dc cultured in 2D and 3D systems were maintained for two (2) days in mESCM without LIF (FIG. 1A, Stage 4). After this point, the samples were cultured under osteogenic conditions for additional 8 and 20 days (FIG. 1A, Stage 5). Control experiments without osteogenic induction were also performed in both 2D and 3D systems by maintaining them in mESCM without LIF during the entire process (FIG. 1A, Stage 5). Then, Ca²⁺ mineralization was assessed as an indication of osteogenic commitment in the 2D and 3D cultures (control and osteogenic) by von Kossa staining, as described in Example 1. In this way, the approximate time of detectable mineralized matrix formation in the cultures could be estimated.

Mineralized nodules became detectable (either 2D and 3D cultures) after approximately 20 days of osteogenic induction, but shorter period of differentiation (8 days) resulted negative for von Kossa staining (not shown). Therefore, the osteogenic time period was set between 20 days and 22 days.

### Example 3

In the experiments in this example, EB-dc in 2D and 3D systems were cultured for two different periods of time in mESCM without LIF before the osteogenic induction. The two time periods were 2 days (Experiment 1) and 8 days (Experiment 2) (FIG. 1A, Stage 4), to determine if a longer period of time at this stage would expand the population of committed cells without affecting its lineage potentiality. The osteogenic time period was set between 20 days and 22 days, as described above in Example 2.

Next, 2D and 3D osteogenic cultures (and controls) were studied by assessing formation of mineralized matrix (von Kossa staining), alkaline phosphatase activity (ALP), and two components of the extracellular matrix deposited by cells undergoing osteoblast differentiation: osteopontin (OPN) and collagen I (Coll I). These techniques have been described above in Example 1.

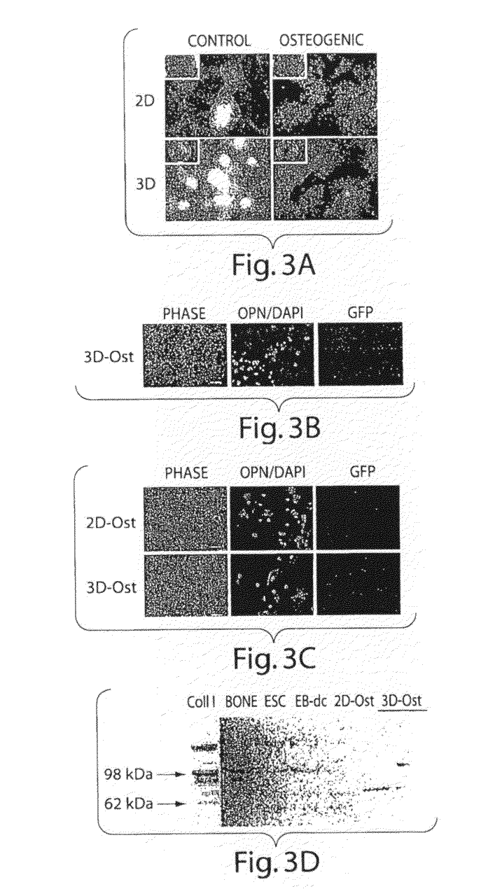

In the second set of experiments, it was found that 2D and 3D cultures without osteogenic induction (controls) stained negative for mineralized Ca²⁺ (FIG. 3A). ALP activity was extensively studied, exclusively in 3D cultures (osteogenic-induced and control), to compare both 2D and 3D systems. Surprisingly, high levels of ALP activity in both osteogenic-induced and control cultures (87 U/mg and 190.2 U/mg, respectively) were observed, indicating that the 3D environment, by itself, induced enzyme activity, independently of the osteogenic conditions. Although ALP is not a specific marker for osteogenesis, its activity may be essential to promote the calcium mineralization process, indicating that in this context the presence of ALP activity is an important factor to consider. In addition, OPN was slightly expressed in the 3D system in a small fraction the cells (FIG. 3B, upper panel) but not detected in the 2D system (not shown). Moreover, Coll I was detected by western blot in both cultures, but more so in the 3D system (FIG. 3D), indicating the enhanced osteogenic phenotype in the 3D system.

More specifically, FIG. 3 illustrates the phenotype of the EB-derived cells after osteogenic induction in 2D and 3D culture system. FIG. 3A shows calcium mineralization staining (von Kossa) after the osteogenic induction of 2D and 3D cultures of EB-dc after 22 days in mESC medium without LIF (Control) or in osteogenic medium (Osteogenic). Mineralized nodules stained black. The scale bar is 100 micrometers. For better visualization of the mineralized nodules, a lower magnification of each well is shown on each top-left corner. FIG. 3B shows osteopontin (OPN) immunofluorescence of isolated cells after osteogenic induction (3D-Ost), in the first set of experiments (EB-dc cultured for 2 days in control medium before osteogenic induction). Cells isolated from 3D osteogenic cultures (3D-Ost) after 22 days in osteogenic medium were assayed for OPN. The scale bar is 50 micrometers. OPN expression was only slightly detected in cells from 3D osteogenic cultures and was not detected in 2D osteogenic cultures (not shown). The expression of GFP was examined under fluorescent microscope.

FIG. 3C shows cells both from 2D and 3D osteogenic induction (2D-Ost and 3D-Ost), in the second set of experiments (EB-dc cultured for 8 days in control medium before osteogenic induction). The cells from both from 2D and 3D osteogenic induction (2D-Ost and 3D-Ost), were isolated after 20 days in osteogenic medium and subsequently assayed for OPN. The expression of GFP was also examined under fluorescent microscope. The scale bar is 50 micrometers. FIG. 3D is a western blot analysis of type I collagen during osteogenic differentiation. Coll I, Collagen-I standard (rat); bone, Mouse bone; ESC, R1 Oct4-GFP ES cells; EB-dc, EB-derived cells; 2Dost, Cells from 2D-cultures after 22 days in osteogenic medium; 3D-Ost, two different samples of cells from 3D cultures in osteogenic medium from the first and second sets of experiments respectively. In general, 3D osteogenic cultures presented higher type-I collagen expression than 2D osteogenic cultures.

Interestingly, it was also found that after 20 days of osteogenic induction, neither 2D nor 3D culture systems were positive for mineralization (not shown) and, in addition, they presented low ALP activity (not shown). Moreover, 2D and 3D osteogenic samples presented high expression of OPN in the extracellular compartment (FIG. 3C), indicating that independently of the osteogenic induction time, these cultures were in an earlier differentiation stage, and therefore suggesting that the period of time that the embryoid body-derived cells were cultured before the osteogenic induction (in this case for 8 days) may have delayed the process of osteogenesis obtaining cells with early osteoblast-like phenotype.

Interestingly in this case, the amount of Coll I detected by western blots was compared to the amount obtained in the first set of experiments. The extracellular matrix protein maintained a marked expression along the osteogenic differentiation, regardless of the delay in the process.

### Example 4

In this example, the proliferation rates of the isolated cells during embryoid body (EB) development were studied, as well as after osteogenic induction and compared to the parental mESC line proliferation rates. The mouse ES cell line cultured in mouse ES medium containing LIF (ESCM) showed a typical exponential growth with an average duplication time (Dt) of 12.6 h (FIG. 2D), characteristic of mouse embryonic stem cells. EB-dc cultured in ESCM without LIF increased their Dt (16.5 h), indicating a decrease in their average proliferation rate during EB development (FIG. 2D). Moreover, average proliferation rates for the total cell population isolated from both osteogenic systems (2D and 3D), sub-cultured in the same medium, decreased to 19.6 h and 32.2 h, respectively, when compared with EB-dc (FIG. 2D). These low proliferation rates, observed in both osteogenic-derived cells, corresponded in time with the appearance of some osteogenic markers, thus suggesting that cultures with higher average duplication times can adopt cell division kinetics more proper of cells that undergo differentiation.

Surprisingly, while performing cell kinetic studies (in mESCM without LIF) it was observed that a small fraction of the cells, either from EB or osteogenic differentiation cultures in 2D and 3D, appear to develop into GFP+ colonies with ESC-like phenotype.

Thus, the frequency of appearance of these GFP+/ESC-like colonies was studied in 2D- and 3D-osteogenic cultures and controls. To do this, the total cells from each culture condition were isolated, counted, and sub-cultured in mESCM with or without LIF. After several days in culture the presence of GFP+/ESC-like colonies was determined and scored (FIGS. 4A-4B) to calculate the frequency of appearance for each condition. FIGS. 4A-4B are examples of ES-like GFP+ colonies observed after the osteogenic differentiation. The bar is 100 micrometers.

Although the frequency for GFP+/ESC-like colonies either in 2D or 3D systems was very low in cultures without LIF (FIG. 4C, −LIF), it was considerably higher for 3D systems cultured in presence of LIF (FIG. 4C, +LIF). Moreover, the 3D system cultured in presence of LIF presented remarkable higher frequency of GFP+/ESC-like colonies than the 2D-system (FIG. 4C, +LIF). This result suggested that, in general, the 3D system culture condition enhanced the maintenance of a small fraction of cells with embryonic-like phenotype compare to 2D-systems. Thus, the 3D system can generate a proper microenvironment conducive to maintaining a small population of undifferentiated cells with pluripotential characteristics. Additionally, these undifferentiated cells remained unaltered after a differentiation process that effectively induced differentiation into osteoblast-like cells, thus, the 3D culture conditions promoted the development of a embryonic stem cell niche.

FIG. 4C illustrates frequencies of ES-like GFP+ colonies found in 2D- and 3D-cultures of EBdc maintained in mESC medium without LIF (control) or in osteogenic medium (osteogenic) for 20 days. The data is expressed as percentage of number of ES-like colonies per 5,000 initial cultured cells. In all cases, cells were isolated from 2D and 3D cultures after the differentiation process and plated in regular 24-well culture dishes at 5,000 cells per well. The cells were grown in ES cell medium (+LIF) and ES cell medium without LIF. ES-like colonies were identified and counted in the microscope. Standard deviations are in parentheses. Frequencies of these ES-like GFP+ colonies are very low. However, the 3D culture system enhanced their presence in comparison with the classical 21) culture system when maintained in the presence of LIF.

### Example 5

The results described in Example 4 with embryonic stem cells suggested expanding these osteogenic differentiation studies to other source of embryonic cells, such as mouse embryonic fibroblast (MEFs), in a 3D system. MEFs were selected as a candidate cell source with potential capability to undergo osteogenic differentiation due to the common mesenchymal origin with adipogenic and chondrogenic lineages.

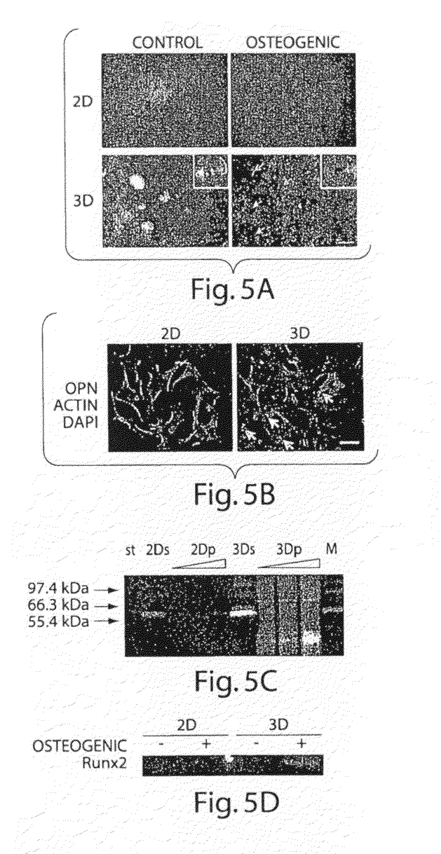

MEFs were encapsulated, using the same peptide scaffold (3D cultures) and maintained in regular MEF medium (control medium) for several days before addition of osteogenic medium (FIG. 1B). MEF 2D and 3D cultures were then switched to osteogenic medium for different time periods (15, 30, and 45 days). It was found that only the MEF 3D cultures were able to develop mineralized matrix by von Kossa staining at time period of 30 days, while 2D cultures did not present mineralized matrix at any time point. This can be seen in FIG. 5A, which shows calcium mineralization of 2D and 3D cultures of MEF after osteogenic induction, for 30 days. Mineralized matrix stains are shown in black. The scale bar is 250 micrometers. A lower magnification of wells of the 3D system is presented in the upper right corner. 2D-osteogenic cultures did not stain for von Kossa, whereas 3D osteogenic cultures showed large number of mineralized nodules (white arrows). 2D and 3D cultures maintained in regular MEF medium (control) were negative for von Kossa.

Alkaline phosphatase activity (ALP) was also evaluated before and after the differentiation process in both 2D and 3D cultures, induced or not. For instance, ALP activity in 2D cultures maintained low values during all the osteogenic induction process: from 1.2 U/mg (initial) to 1.6 U/mg (after 30 days induction). Similar values were observed in 2D control cultures: from 1.44 U/mg (initial) to 1.66 U/mg (after 30 days of culture), suggesting that the 2D system did not induce ALP activity and therefore provide low osteogenic potential to the culture. Instead, ALP activity in 3D osteogenic cultures dramatically increased after osteogenic induction (from 1.5 U/mg to 118.3 U/mg) as well as in controls (from 1.8 U/mg to 68.1 U/mg), indicating that the 3D environment was instructive enough to induce ALP activity independently of the osteogenic conditions, as previously described for the mESC system (see above). Calcium mineralization (von Kossa staining) and ALP activity results from 2D and 3D cultures suggested that not only specific osteogenic supplements are needed to promote the osteogenic commitment of MEF but also a 3D environment appeared to be important. Finally, expression of the transcription factor Runx2 and Collagen I (Coll I) was analyzed to assess the level of osteogenic commitment in 2D and 3D systems. Only the 3D conditions appeared to expressed Coll I (not shown) and more specifically, only the 3D osteogenic condition appeared to expressed Runx2 (FIG. 5D), indicating their strong osteogenic commitment obtained in this culture condition. It is important to mention that the osteogenic commitment in this case did not required the addition of specific growth factors such as Bone Morphogenic Proteins (BMPs), known to promote osteogenesis. Thus, the MEF system presented a more homogeneous population of cells, and were easier to culture, expand, and perform differentiation protocols (FIG. 1B).

### Example 6

In the above examples, the potential of MEFs to enter a differentiation program directed to the formation of osteoblast-like cells was demonstrated. Based on those examples, and the fact that high ALP activity can be induced in a 3D culture environment, in this example, MEFs cultured in 3D under control conditions were shown to have some properties of immature osteoblastic cells. Here, OPN expression was evaluated prior to osteogenic induction in both 2D and 3D cultures, since it is described as an osteoblast marker proper of an early stage of osteoblast differentiation.

MEFs cultured in a 3D system and maintained for 15 days in their regular medium (MEF medium) were immunoreactive to OPN (FIG. 5B). However, 2D cultures of MEF at the same time did not express OPN (FIG. 5B). Interestingly, cells from the 3D cultures presented perinuclear OPN expression but not in the extracellular matrix, suggesting that the OPN was synthesized but not secreted, presumably stored in granule-like structures (FIG. 5B). This result suggested that MEFs cultured in a 3D system acquired a distinct stem cell-like phenotype, which may be a prerequisite for their marked osteogenic potential in a 3D system.

More specifically, in FIG. 5B, which illustrates osteopontin (OPN) immunofluorescence of MEF cultured in 2D- and 3D-culture systems, MEF were cultured in 2D and 3D systems for a period of 15 days with regular MEF medium. After this time period, MEF from 3D system were isolated and plated in order to perform the immunofluorescence analysis of osteopontin. MEF of the 2D system were analyzed as controls. The staining was as follows: anti-OPN immunofluorescence (FITC, green, indicated by white arrows), F-actin staining (Rhodamine, red, predominately in intracellular regions outside the nuclei), and DAPI staining for nuclei (blue, predominately in nuclear regions). OPN-positive zones are marked by white arrows. The scale bar is 50 micrometers.

### Example 7

In this example, metalloproteinase activity of the potential “stem cell-like” intermediate obtained in the 3D cultures were analyzed to determine if the 3D culture recreated some of the aspects of an embryonic-like regenerative model of wound healing. Wound healing proceeds with the formation of a blastema, scar-less type, and with the replacement of lost tissue such as skin and cartilage by normal functional tissue. Thus, in an embryonic-like model, the breakdown of the extracellular matrix (ECM) by metalloproteinases (MMPs) secreted by dermal fibroblast to create a regenerative blastema structure may be a critical event.

MMP-2 and MMP-9, known as type IV and V collagenases or 72 kDa gelatinase A and 92 kDa gelatinase B, respectively, are secreted by migrating and proliferating fibroblast, therefore reducing the amount of ECM and basement membrane prior to a blastema development, as has been previously described during the regeneration of ear defects in the MRL mouse. In addition, osteopontin (OPN) is involved in normal tissue remodeling process such as bone resorption, angiogenesis and wound healing. Moreover, OPN induces pro-MMP-2 and pro-MMP-9 activations by two distinct pathways. First, OPN induces nuclear factor κB-(kappa-B) (or NFκB, NF-kappa-B) mediated pro-MMP-2 activation though IκBα/IκBα (1-kappa-B-alpha/1-kappa-B-alpha) kinase (IKK) signaling pathway. Second, OPN induces αvβ3 (alpha-V-beta-3) integrin-mediated phosphorylation and activation of nuclear factor-inducing kinase (NIK) and NIK then induce pro-MMP-9 activation through MAPK/IKKα/β (MAPK/IKK-alpha/beta) mediated pathway, and all these control cell motility, invasiveness, and eventually various aspects of the wound healing process. Thus, for these reasons expression of OPN and MMPs may be important markers to consider in this MEF system.

In this example, culture supernatants and cell fractions were obtained from both 2D and 3D cultures before osteogenic induction, and the presence of metalloproteinase activity by zymography (with gelatin) was assessed. Initially, the presence of MMP activity in the culture medium was detected, due to the fetal bovine serum (FBS) used to prepare the medium (FIG. 5C, “M”). Since the presence of this background metalloproteinase activity from the FBS complicated the analysis of the culture supernatants because of the superposed MMP activity secreted by the cells from 2D and 3D systems (see FIG. 5C, “2Ds” and “3Ds,” respectively), it was decided to study the presence of MMP activity directly from the cell extracts.

In FIG. 5C, which illustrates zymography showing matrix metalloproteinase (MMP) activities from MEF maintained in their regular culture medium in 2D and 3D culture systems, abbreviations are as follows: st, molecular weight standard Mark12 (Invitrogen); 2Ds, medium supernatant from MEF cultured in 2D (4 days old); 2Dp, three cell extract samples of 2D-cultures of MEFs (4 days old) of increased protein concentration; 3Ds, medium supernatant of MEFs cultured in 3D (20 days old); 3Dp, three cell extract samples of 3D-cultures of MEFs (20 days old) of increased protein concentration; and M, samples of regular MEF culture medium.

Bands at 72 kDa and 62 kDa, in three samples of increasing concentrations of proteins from cell extract of 3D cultures, may correspond to proMMP-2 and active MMP-2 respectively (FIG. 5C, “3Dp”). In the same lanes, some activity was also detected at higher molecular weights (˜92 kDa), which corresponded to MMP-9 (FIG. 5C, “3Dp”). The bands seen at lower molecular weights in the same lanes have not been identified but may correspond to intracellular proteolytic activity of the 3D system. Interestingly, in lanes corresponding to the cell extract activity of the 2D system, only the active form of MMP-2 was slightly detected (FIG. 5 C, “2Dp”). Hence, it can be concluded that during the period of 3D culture before osteogenic induction, MMP-2 and MMP-9 metalloproteinase activities were up-regulated, mainly in MEFs 3D-cultures (FIG. 5C).

Moreover, MEFs isolated from the 3D system acquired a distinctive phenotype of small, elongated cells, which notably differed from the phenotype of MEFs grown in 2D-system (not shown). However, this distinctive phenotype was not maintained after these 3D-derived cells were cultured in regular (2D) culture dishes for several days, becoming morphologically similar to MEFs initially cultured in 2D. This result indicated that the maintenance of the phenotype was strictly dependent on the 3D environment.

Finally, in terms of proliferation capacity, MEFs isolated from 3D system after 15 days of culture and plated in regular culture dishes maintained their initial proliferation capacity, with a duplication time of ˜47 h (not shown). In contrast, MEFs cultured in regular plates became senescent after a period of approximately 20 days.

In conclusion, the 3D culture system provided a cellular microenvironment that promoted MEFs transition into a “stem cell-like” phenotype with markedly metalloproteinase activity and persistent mitotic activity, characteristic of regenerative mesodermal tissues, such as blastema fibroblast.

### Example 8

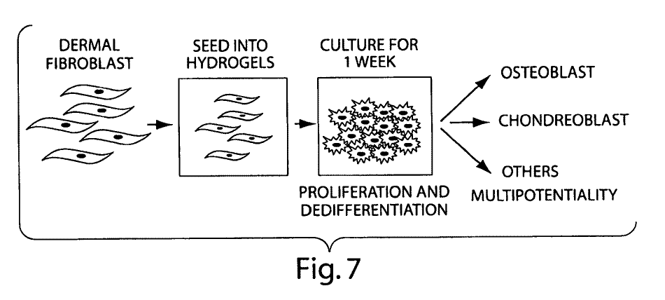

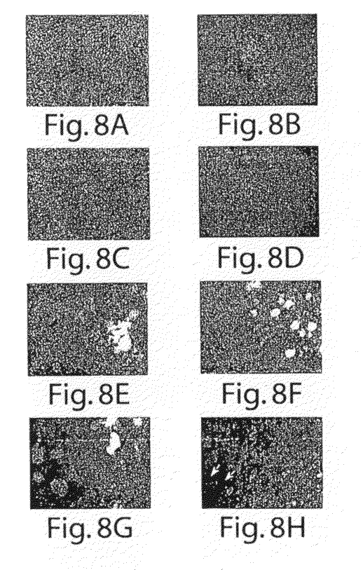

The above examples show that the intervention of a non-regenerative wound can be induced to regenerate if provided with an adequate microenvironment. This microenvironment can be made of a three-dimensional nanofiber scaffold that mimics the blastema milieu inducing proximal cells to engage in a regenerative response. In the above examples, it was observed that by simply growing mouse dermal fibroblast in self-assembling peptide scaffolds for a week, they can turn into a multi-potential phenotype by up-regulating the expression of two adult stem cell markers, osteopontin and nestin (FIG. 7). At this stage, the dedifferentiated cells appeared to possess the capacity to differentiate into osteoblast and chondreoblast cell type. After this, the cells were found to have differentiated into osteoblastic phenotype (von Kossa staining for calcium mineralization) or chondroblastic phenotype (DMMB stain for proteoglycans), respectively. It was also demonstrated that mouse dermal fibroblasts were able to turn into a multipotential cell-type after culturing them into a three-dimensional synthetic scaffold system. When exposed to osteogenic media, the fibroblasts differentiated into osteoblast-like cells (FIG. 8).

FIG. 8 illustrates Von Kossa staining of mouse embryonic fibroblast (MEF) cultured in regular culture dishes (FIGS. 8A-8D) or in three-dimensional peptide scaffolds (FIGS. 8E-8H). FIG. 8A is a phase contrast of MEFs cultured in control non-osteogenic media (4 weeks); FIG. 8B is the same optical layer with transmitted light to detect the von Kossa staining. FIG. 8C is a phase contrast of MEFs cultured in osteogenic media (4 weeks); FIG. 8D is the same optical layer with transmitted light to detect the von Kossa staining. FIG. 8E is a phase contrast of MEFs cultured in control non-osteogenic media (2 weeks). FIG. 8F is phase contrast of MEFs cultured in osteogenic media (2 weeks). FIG. 8G is phase contrast of MEFs cultured in control non-osteogenic media (6 weeks). FIG. 8H is phase contrast of MEFs cultured in osteogenic media (6 weeks). Bar correspond to 200 micrometers. White arrows in FIG. 8H indicate examples of positive von Kossa staining (black precipitates).

### Example 9

This example demonstrates that primary mouse embryonic fibroblasts (MEFs) can become mesenchymal multipotent after culturing them into 3D-envitonments. Other examples herein have shown that, after culturing primary mouse embryonic fibroblasts into a three-dimensional self-assembling peptide scaffold for several days, the fibroblasts can upregulate osteopontin and differentiate into an osteoblast-like cell after induction in osteogenic medium. The cells in the 3D-osteogenic cultures presented a phenotype proper of a system that underwent differentiation into osteogenic lineage including presence of calcium mineralization, up-regulated alkaline phosphatase activity, collagen type I synthesis.

In these examples, the acquisition of the “multipotent” state may be caused, at least in part, to the three-dimensional matrix that promoted the cells to undergo into a mesenchymal progenitor cell-like with capacity to differentiate into the osteoblast lineage. In order to explore in more detail the effect that a 3-dimensional environment cause on the cells, in this example, MEFs were initially cultured into two different three-dimensional nanofiber scaffolds systems including agarose and Collagen I gels to determine whether or not these two chemically unrelated matrices (a polysaccharide and a protein base gel material) promoted the acquirement of mesenchymal potentiality observed before in self-assembling peptides scaffolds. In addition, in this example, the expression of not only osteopontin but also nestin in the 3D cultures were analyzed. In addition, mesenchymal potentiality was studied by inducing osteogenesis as well as chondrogenesis and adipogenesis.

2×10⁶ MEF/ml were encapsulated into 0.25% of agarose gels, 0.2% collagen I gels and 0.25% of self-assembling peptide gels and cultured in DMEM (high glucose) with 15% FBS for several days. Then, cells were isolated from each 3D system after 5 and 10 days of culture, and stained for nestin and osteopontin. The two progenitor markers were observed to be up-regulated in a high percentage of the cell population (between 20-50%) of the cells across all the systems, suggesting that any of the 3D systems, regardless of their chemical nature, may induce a progenitor-like phenotype.

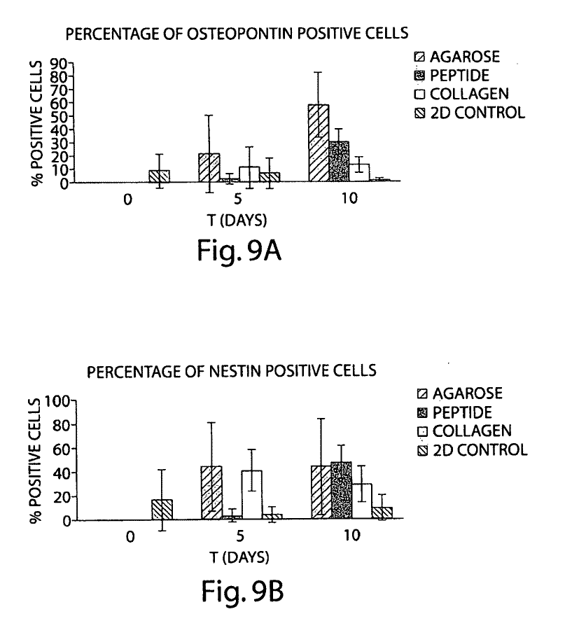

FIG. 9 illustrates primary mouse embryonic fibroblast cultured in 3D scaffolds upregulated with nestin and osteopontin. MEFs cultured in different 3D-scaffolds (agarose, collagen I and self-assembling peptide gels) with fibroblast media (DMEM with 15% FBS) for different times (0-10 days) were isolated from each gel type and plated overnight in regular culture dishes. 2D control cultures were also performed. Then, expression of two progenitor cell markers were assessed by immunofluorescence, positive cells for each marker were counted and percentage were calculated based in total amount of cells for each condition and time. FIG. 9A shows cells were stained for osteopontin and FIG. 9B shows cells stained for nestin.

To confirm that the mesenchymal potential capacity that each 3D-system acquired, after 10 days of culture in fibroblast medium, the cells were exposed (including the 2D controls) to osteogenic, chondrogenic, or adipogenic induction medium to determine if the cells would differentiate respectively in those lineages. As expected, the 3D-systems, but not the induced 2D controls, differentiated in their respective mesenchymal lineage after induction, as shown in Table 1. In this table, the abbreviations are as follows: FM: Fibroblast medium (control medium), Osteo: osteogenic medium, Chondro: chondrogenic medium, Adipo: adipogenic medium; nt: not tested. Osteogenesis was detected by Calcium mineralization (von Kossa staining), chondrogenesis was detected by deposition of Glycosaminoglycans such as Agrecan (Toluidine blue staining), and adipogenesis was detected by cell morphology (big lipid vesicules) and lipid staining (Nile red staining).

TABLE 1

| Staining | Osteogenesis | | Chondrogenesis | | Adipogenesis | |
|---|---|---|---|---|---|---|
| Medium | FM | Osteo | FM | Chondro | FM | Adipo |
| 2D | − | − | − | − | − | − |
| Peptide | − | +++ | + | nt | + | nt |
| Agarose | − | +++ | − | − | − | +++ |
| Collagen I | − | +++ | − | + | − | +++ |

These results show that the 3D environment, under the culture conditions used, and regardless of their chemical nature (polysaccharide, self-assembling peptide fiber, or extracellular matrix protein), can be sufficient for induction of MEFs into a multipotent mesenchymal progenitor-like cell. Thus, the cells “sense” the environment and can be “reprogrammed” into a multipotent progenitor, suggesting that for regenerative purposes, a three-dimensional scaffold can be used.

### Example 10

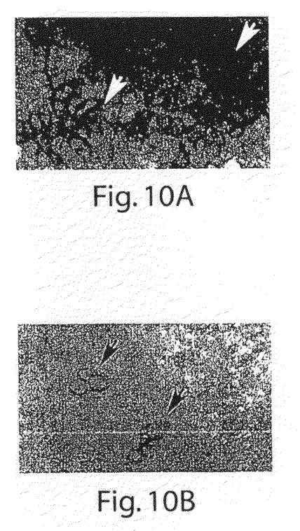

This example illustrates that MEFs can undergo distinct morphological changes in special scaffold conditions. By examining at the results present in Table 1 chondrogenesis and adipogenesis can be observed to also occur with MEFs cultured with fibroblast media (control) in self-assembling peptide scaffolds only, indicating that this system, in particular, may promote a default differentiation process into these lineages, which may be caused by creating a cell microenvironment (Table 1) (FIG. 10).

Thus, in this example, differences between the systems were closely studied. Collagen I gels, agarose gels, and self-assembling peptide gels were observed to show morphological changes of contraction during the culture. Fibroblasts cultured in collagen I gels can contract the matrix to reduced size structures. The contraction observed in the self-assembling peptide system suggested also that in both systems the cells are in similar biomechanical conditions. However, in the collagen I system the cells did not undergo natural chondrogenesis after culturing them with fibroblast media (Table 1). Since collagen I is the natural extracellular matrix component of the dermis and the bones, this could indicate that this material may be instructive in guiding the cells into dermal or osteogenic lineages, which may prevent them from spontaneous differentiation into chondrogenic lineages (Table 1). Thus, this may be an example of cellular instruction by the collagen I matrix in comparison with the self-assembling peptide scaffolds that, per se, do not have a designed signaling capacity. In addition, it also suggested that the absence of signaling (or chemical instruction) could be a cause for these gels to allow cells to naturally undergo chondrogenic or adipogenic lineage differentiation (FIG. 10).

In FIG. 10, natural chondrogenesis and adipogenesis of MEFs cultured in control self-assembling culture conditions is shown. In this figure, MEFs were cultured in self-assembling peptide gel for 30 days in fibroblast media (DMEM with 15% FBS). FIG. 10A shows toluidin blue staining to detect aggrecan deposition in the extracellular matrix, while FIG. 10B shows phase contrast to detect adipocyte morphology. White arrows indicates positives clusters of aggrecan and black arrows indicates clusters of adipocytes with big lipid vesicles.

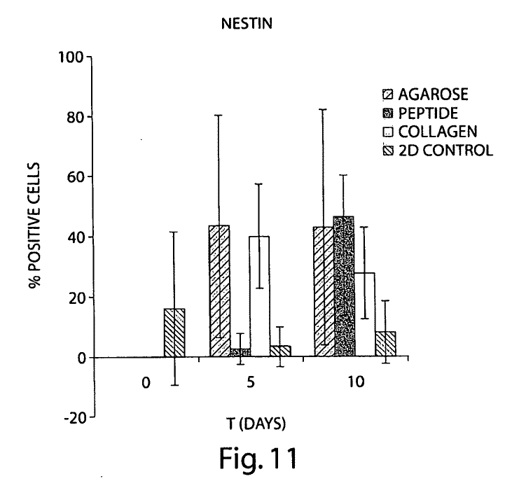

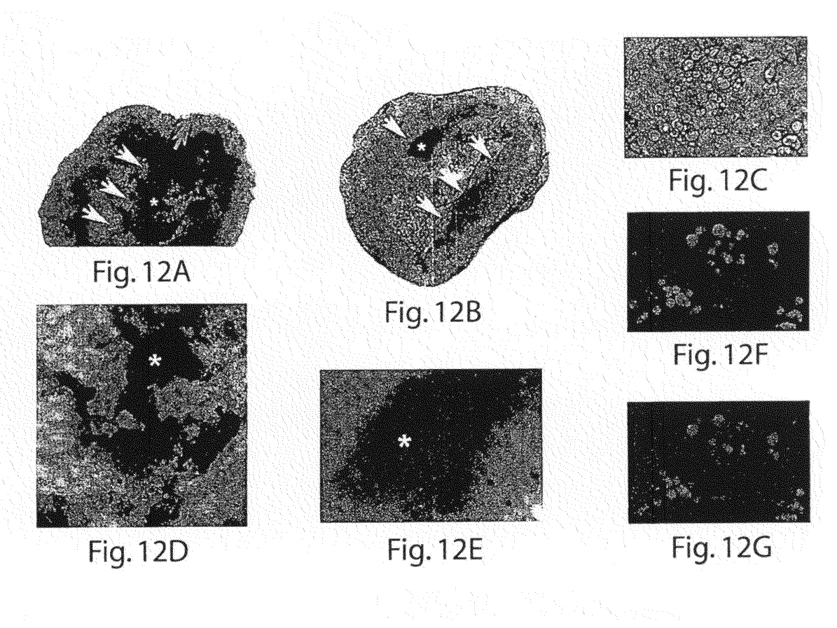

In order to explore in more detail the effects of a 3-dimensional environment on cell behavior Mouse Embryonic Fibroblasts (MEFs) were cultured into three 3-dimensional nanofiber scaffolds: the self-assembling peptide RAD16-I, Agarose and Collagen I gels (a polysaccharide and two protein based gel materials). The expression of nestin (a progenitor cell marker) was also analyzed in the cultures. 2×10⁶ MEF/ml were encapsulated in 0.25% of Agarose gels, 0.2% Collagen I gels and 0.25% of self-assembling peptide gels and cultured in fibroblast medium (FM) for several days. Cells were then isolated from each 3D-system after 5 and 10 days of culture and stained for nestin. The progenitor marker nestin was up-regulated in a high percentage of the cell population (between 20-50% of the cells) across all the systems, suggesting that any of the 3D systems are adequate to induce a progenitor-like phenotype (FIG. 11A). In order to confirm the mesenchymal potential capacity that MEFs acquired in each 3D-system (and 2D controls), after 12-days of culture in fibroblast medium the cells were exposed to osteogenic, chondrogenic or adipogenic induction medium to assess lineage differentiation. Only the 3D-systems, but not the 2D systems (induced and controls), differentiated into the three mesenchymal lineages (FIG. 12 and Table 2). For instance, in the case of osteogenesis, the cells stained positive for calcium mineralization in all 3D-systems, but only after induction (FIG. 12 and Table 2). Interestingly, adipogenesis occurred in all the 3D-systems both in control medium (FM, fibroblast medium) and adipogenic medium. Chondrogenesis, however, arose only in the self-assembling peptide scaffolds, of those tested (FIG. 12 and Table 2).

It was discovered that the collagen I gels and the self-assembling peptide gels, both experienced morphological changes such as gel contraction during the culture of the cells. As a result, the size of the 3D-construct was reduced several fold. It is known that fibroblasts contract the matrix to reduce dermal tissue size during wound healing. The contraction observed herewith the self-assembling peptide system suggested that in both systems the cells undergo similar biomechanical stress. However, in the collagen I system, the cells didn't undergo natural chondrogenesis after being cultured in control medium (Table 2).

TABLE 2

| Staining | Osteogenesis | | Chondrogenesis | | Adipogenesis | |
|---|---|---|---|---|---|---|
| Medium | FM | Osteo | FM | Chondro | FM | Adipo |
| 2D | − | − | − | − | − | − |
| Peptide gel | − | +++ | + | ++ | + | ++ |
| Agarose | − | +++ | − | − | + | + |
| Collagen I | − | +++ | − | − | + | + |

Surprisingly, when the morphological changes of the self-assembling peptide gel system were analyzed in more detail, in contrast to the collagen I gel, the self assembling peptides were observed to develop into a structure with a central axis and two thick parallel structures at both sides of the central line (FIG. 12 panel a and b). Moreover, it was observed that only under one of the tested culture conditions (self-assembling peptide scaffold stiffness and fetal bovine serum concentration) the system underwent cellular self-organization developing into a 3D-bilateral structure, indicating that the biomechanical and biological initial parameters are important (Table 3). The shape presents bilateral symmetry (3D-bilateral) and resembles some aspects of a vertebrate embryo undergoing axis formation. Collagen I cultures presented a different morphology, basically consisting of a small spherical ball produced by equal contraction forces, without any distinctive shape or pattern. Interestingly, after osteogenic induction of the 3D-bilateral structure obtained in the peptide gel system, a von Kossa positive zone (Calcium mineralization) was observed at the central line, suggesting that this area is sensitive to induction (FIG. 12 panel a and d). Additionally, when the 3D-bilateral structures were stained with Toluidine blue for glycosaminoglycans (GAGs), two positive zones parallel to the central axis were detected, suggesting the development of a paraxial cartilage-like tissue (FIG. 12 panel b and e).

FIG. 12 shows a phenotype assessment under different culture conditions in 3D-self-assembling peptide hydrogel RAD16-I such as after osteogenic differentiation and von Kossa staining (panels a and d); or Toluidine blue staining of MEF after default chondrogenic differentiation in control cultures (panels b and e); or after assessing adipocyte presence in control cultures by visual inspection (panel c, phase contrast) or after Nile red staining to detect neutral lipids (panel f fluorescein filter; panel g, rhodamine filter).

TABLE 3

| | % FBS - 2 | % FBS - 10 | % FBS - 15 |
|---|---|---|---|
| % RAD - 0.25 | No contraction | Low | High contraction; |
| | | | Formation of |
| | | | bilateral structures |
| % RAD - 0.5 | No contraction | Very low | Low |

Thus, localized chondreogenic induction that developed in the system may be the result of mesenchymal progenitor differentiation under the control of an early-organized mesodermal process that directs localize and patterned differentiation. Thus, the system may be “recapitulating” development in a self-organized fashion, creating an embryoid-like structure, i.e., an “embryoid,” with mesodermal lineage multipotential.

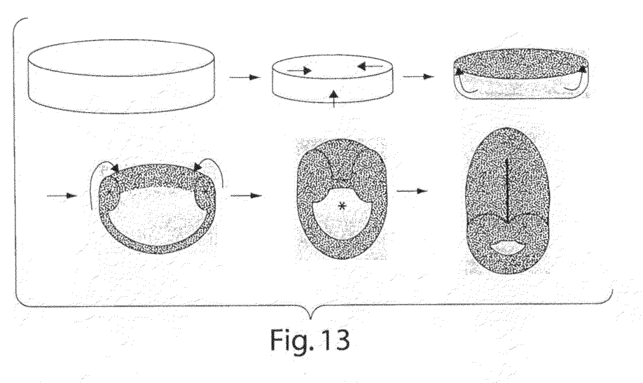

The morphological development of the 3D-bilateral structure was examined over time. During the first days in culture the cells contracted the scaffold from a disc-shape to a much smaller flat dense disk. Then, in the following days the edge of the disk continued contracting producing a compaction of its perimeter and turning it into a wheel-like shape with semicircular cross-section or dome shape. Next, two diametrically opposite zones at the edge of the wheel or dome started actively contracting inward merging at the center, compressing both sides. As a consequence, the compaction of the cell masses from each side of the dome caused a merging line zone that forms a “middle line”. This process elongates the body along the axis with two large and dense paraxial structures, resulting in a 3D-bilateral assembly. At this point the cell mass has gone through the main morphological changes. A model suggesting the main morphological changes that the system undergoes to develop into a 3D-bilateral structure is shown in FIG. 13. Interestingly, optical cross-sections at two time points clearly evidenced the formation of an internal cavity as a result of this morphogenetic process (FIG. 13).

FIG. 13 shows morphogenesis of MEF in soft self-assembling peptide cultures. A model indicating the main morphological processes is presented as a guide to help understanding the development of the 3D-bilateral structure. The asterisks indicate also the zone of lateral force generation and the presence of a cavity developed during the morphological process and the empty cavity present in the structure.

### Example 11

This example shows the control of various external parameters to promote cellular self-organization. The percentage of self-assembling peptide used for these experiments was half of the amount we described above (0.25% vs. 0.5%). Stiffer materials did not apparently result in such morphological changes. Thus, in this example, two different parameters that may affect the development of such embryoid-like structures were studied: self-assembling peptide scaffold stiffness and fetal bovine serum concentration. These results are presented in Table 4 In these experiments, MEFs were cultured in self-assembling peptide scaffolds (2×10⁶ cells/ml) at two peptide concentrations (0.25% and 0.5%) for 10 days in fibroblast medium (DMEM high glucose with 2 or 15% of fetal bovine serum, FBS). Gel contraction and the presence of embryoid-like structure development were analyzed. nt=condition not tested. These results indicate that the particular biomechanical environment (microenvironment) may be at least partially responsible for the generation of the embryoid-like structures.

TABLE 4

| Peptide gel % | FBS % | Gel contraction | Embryoid-like* |
|---|---|---|---|
| 0.25 | 2 | Low | − |
| 0.25 | 15 | High | + |
| 0.5 | 2 | nt | nt |
| 0.5 | 15 | Low | − |

The chronology of the morphological changes in the conditions that allow the development of an organized embryoid-like structure were then studied (Table 4). MEFs cultured in self-assembling peptide gels (at 0.25%, DMEM with 15% FBS) were monitored during 10 days of culture. It was observed that during the first two days of culture, the cells contracted the scaffold from a disc-shape gel to a flat dense disk, several times smaller. Then, in the following two days, the edge of the disk continued contracting, producing an engrossment of its perimeter and turning it into a wheel-like shape, with a semicircular cross-section or dome shape. Next, around day 5-7, two diametrically opposite zones at the edge of the wheel or dome started actively migrating outward of the structure, merging at the top of it compressing from both sides. As a consequence, the migration of the cell masses from each side of the dome caused a merging zone that formed a middle valley or streak along the axis, elongating the body along the axis with two large and dense paraxial structures. Depending where exactly the lateral forces start they produced two types of final structures: a linear middle streak or a bifurcated (Y-like shape) middle streak. At this point, the structure appeared to have gone though the main morphological changes, as the structure remained morphologically similar, at least, for the next 20 days.

### Example 12

This example illustrates mesodermal induction and pattern formation. In this example, the possibility that the embryoid structures are engaged in recapitulating some stages of early development, presumably mesoderm induction, is studied, mainly based in the lineage origin of dermal tissues. The expression pattern of the transcription factor brachyury, which is an early marker during notocord development and mesoderm induction, was studied.

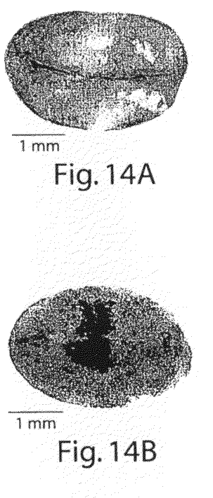

Interestingly, at day 7, brachyury was evident in the middle streak zone of the embryoid, in particular staining a defined zone where the two migratory cell masses had merged (FIG. 14A). In early vertebrate development, brachyury is expressed first in the presumptive notocord and then is evident in the early mesoderm, at the both sides of the primitive streak. Furthermore, by day 11 of culture, brachyury positive staining extended into the entire paraxial structure or the embryoids suggesting that, analogous to embryogenesis, the mesodermal induction continues advancing at the both lateral structures, in a way that resembles a presumptive paraxial mesoderm (FIG. 14B). In FIG. 14, the Embryoid-like structures at day 7 (FIG. 14A) and at day 11 (FIG. 14B) were immunostained for brachyury using a primary antibody anti-brachyury, developed with a secondary antibody-HRP conjugated. The pictures were taken from the top of the dome where a clear primitive streak-like can be observed along the middle axis.

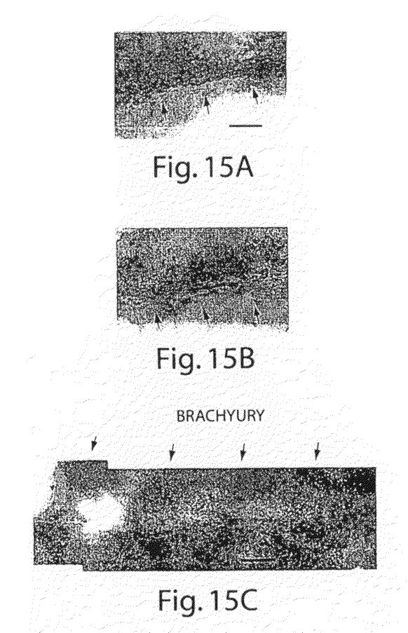

The expression of brachyury in both samples was analyzed in more detail with higher magnification. At day 7, positive cells were localized at the edges of the merging zone, in a way that resembles notocord structure development (i.e., like a tube development), just at the bottom plaque of the middle streak-like structure (FIGS. 15A-15B). Later, at day 11, not only clear expression was extended a both sizes of the middle streak, but also the cells were organized in groups along the axis, in a way that resembled somite formation (FIG. 15C). This suggested that the mesoderm in the embryoids was induced along the axis. It also showed self-organizing a pattern of expression, with clear and defined groups of cells forming clusters in a way that resembled somites (FIG. 15C). This suggested that the system underwent segmentation, a fundamental process during development where cells in embryonic tissues position themselves to control the formation of the main body plan. In addition, this is consistent with the segmentation observed previously in the paraxial chondrogensis produced in the 30-day old embryoid-like structures, as previously described.

FIG. 15 illustrates embryoid-like structures at day 7 (FIGS. 15A-15B) and at day 11 (FIG. 15C), immunostained for brachyury using a primary antibody anti-brachyury, developed with a secondary antibody-HRP conjugated. The magnification used here revealed cell organization during mesoderm induction. Black arrows in FIGS. 15A and FIG. 15B indicated that a tubular-like structure developed early (presumably an early notocord-like structure) and in FIG. 15C, the segmentation on the dorsal part composed by positive cell groups at both sides of the axis, presumptive somites and the origin of segmentation and body plan.

To confirm the segmentation observed in embryoids of 11 days, the structures were stained them for MyoD, an early myogenic marker that is express during somitogenesis in a pattern along the central axis. Since the expression of MyoD evidenced the first muscle progenitors, the early mesodermal induction observed could also promote the subsequent development of myoblast, eventually in an organize pattern as well. Again here, a pattern of expression was observed in an embryoid of 30 days, indicating that presumably early myoblast cells were generated in clusters similar to somites, following a body segmentation plan. The positive stained cell clusters, presumably early myoblasts, follow a pattern that suggests an early organization by segmentation along the axis.

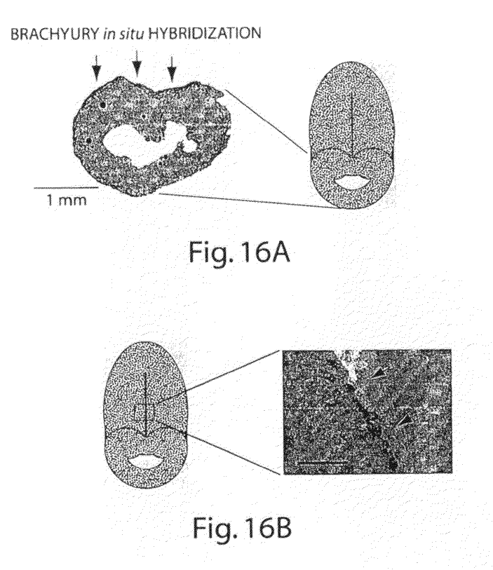

The localization of Brachyury expression was confirmed by in situ hybridization. Probes were prepared with the same primers used for regular RTPCR and after the staining we observed that the Brachyury mRNA was mainly expressed at the same zone detected with immunohistochemistry, confirming the expression of this early organizer transcription factor in the 3D-bilateral structures (FIG. 16 panel b). Staining of a cross-section of this structure depicts the clear presence of an internal cavity and the expression of Brachyury at the external paraxial zone (FIG. 16 panel a). FIG. 16 shows molecular characterization of the mesodermal induction process observed in MEFs cultured in RAD16-I cultures. In situ hybridization was also performed to observe the localization of Brachyury. Totally contracted samples (15 days of culture in FM) were fixed and cryosectioned as indicated. In situ hybridization over a 14 μm slice of the cross-section obtained is shown in panel a. Short after closure of the central axis (11 days of culture), the cell mass was fixed. Whole mount in situ hybridization showed the localization of Brachyury (panel b).

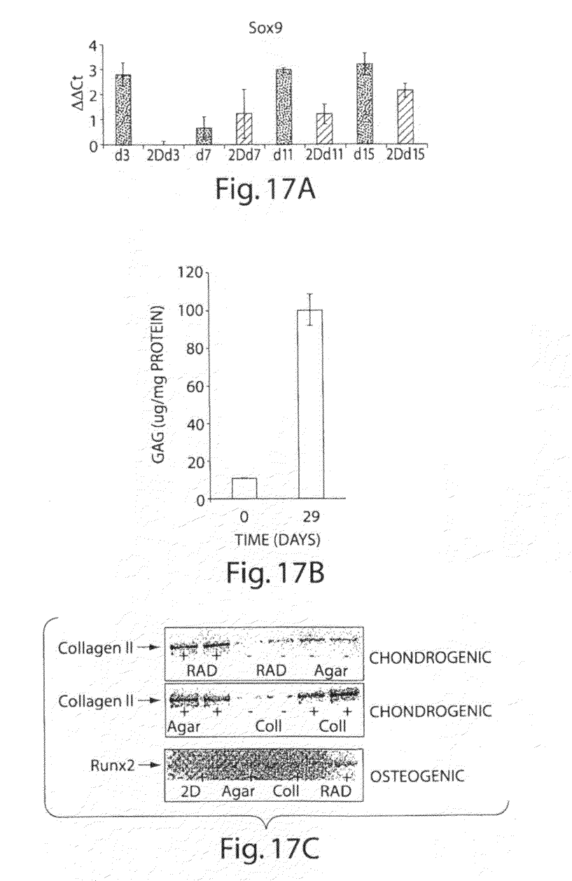

Expression of Sox9, Collagen-II, and Runx2 as well as presence of GAGs was analyzed to assess the level of chondro-osteogenic commitment in 2D- and 3D-systems. The expression of the transcription factor Sox9 was upregulated overtime mainly in the self-assembling peptide scaffold system, suggesting a strong chondrogenic commitment around day 11 and 15 of culture (FIG. 17 panel a). After an osteogenic differentiation protocol, the expression of the transcription factor Runx2 was analyzed by western blot in 2D- and in all 3D-systems: agarose gels, self-assembling peptide scaffolds, and collagen I gels (FIG. 17 panel c). Then, in order to describe in more detail the presence of cartilage-like tissue we studied the presence of other molecular markers including Collagen type II and GAGs. Collagen type II was upregulated after a chondrogenic differentiation protocol in all the 3D-systems (FIG. 17 panel c). In addition, GAGs were studied in fibroblasts, cultured in the self-assembling peptide with FM, and its production increased overtime, confirming the default cartilaginous commitment of these cells (FIG. 17 panel b).

FIG. 17 shows molecular characterization of the mesodermal induction process observed in MEFs cultured in RAD16-I cultures, quantitative Real Time PCR (RT-PCR) of Sox9 transcription factor. Total mRNA was isolated from MEFs cultured in RAD16-I with FM, and from 2D controls, after days 3, 7, 11 and 15. (panel a). Glycosaminoglycans (GAGs) from 3D-self assembling peptide cultures of MEF at days 0 and 29 were quantified with DMMB (1,9-dimethyl-dimethyleneblue) using chondroitin sulphate as standard (panel b). Collagen type II and Runx2 expression was analyzed by western blot in MEFs after a chondrogenic (collagen-II) or osteogenic (Runx2) differentiation protocol. Agarose, collagen-I and RAD16-I were used as 3D-environments for MEFs; 2D cultures were used as control (panel c).

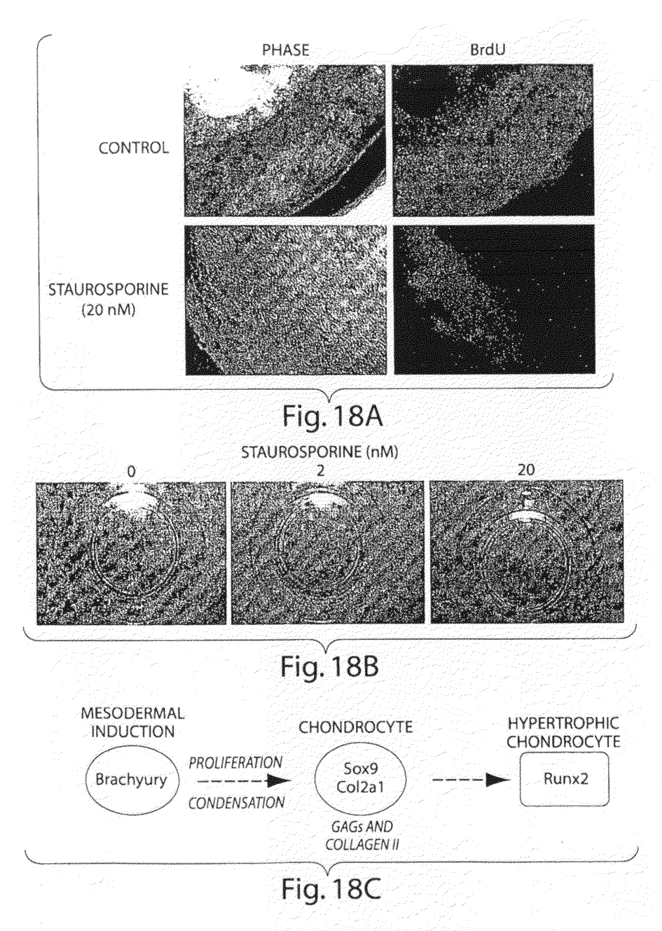

Finally, Staurosporine (an inhibitor of the Protein Kinase C, that arrests cells in G1) treatment clearly abrogated the development of the 3D-bilateral structure (FIG. 18 A). In addition, no incorporation of BrdU was detected in Staurosporine-treated samples. Moreover, a dose-response inhibitory effect on the 3D-bilateral structure development was observed when diluted concentrations of Staurosporine were added to the cultures (FIG. 18B). FIG. 18 shows inhibition of the 3D-bilateral structure development by cell cycle arrest induced by staurosporine. (A) MEFs were incubated with and without staurosporine (20 nM) during 7 days. Proliferation was studied by means of a BrdU pulse followed by immunostaining against BrdU. (B) MEFs were incubated, during 14 days, with different concentrations of staurosporine (0, 2, 20 nM) to further analyze the effect of proliferation in the contraction phenomenon. (C) Finally, we propose a mesodermal commitment and cartilage-like tissue development model in our system based in the molecular markers detected.

Thus, these examples demonstrate a process that recapitulates early development in vitro using mouse embryonic fibroblast cells of dermal origin in a determined biomechanical 3D-environment. This process included the acquisition of multipotentiality and cellular self-organization. Thus, these systems may autonomously progress into a stage that induces a developmental program that promote not only embryonic-like mesoderm induction but also an early body plan or segmentation. Since the system recapitulates some aspects of development, this indicates that dermal fibroblast, after turning into a multipotent progenitor cell type, may engage into a process similar to what dermal fibroblast undergo during amphibiam limb regeneration: development of a blastema structure where dermal fibroblast go through mutlipotentiation and mesodermal redevelopment.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. All definitions, as defined and used herein, should be understood to control over dictionary definitions, definitions in documents incorporated by reference, and/or ordinary meanings of the defined terms. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to.

Only the transitional phrases “consisting of” and “consisting essentially of” shall be closed or semi-closed transitional phrases, respectively.

Each of the references and patents and applications described herein is incorporated by reference, including U.S. Ser. No. 60/809,908 filed by the same inventors on Jun. 1, 2006, the entire contents of which is incorporated by reference.

What is claimed is:

1. A method for producing a stem cell phenotype, comprising:
culturing fibroblasts within a three-dimensional matrix in the presence of an anti-inflammatory factor to produce a stem cell phenotype.

2. The method of claim 1, wherein the matrix comprises a peptide scaffold.

3. The method of claim 1, wherein the matrix comprises a peptide hydrogel.

4. The method of claim 1, wherein the matrix comprises a self-assembled peptide.

5. The method of claim 1, wherein the matrix comprises a polysaccharide.

6. The method of claim 1, wherein the matrix comprises agarose.

7. The method of claim 1, wherein the matrix comprises alginate.

8. The method of claim 1, wherein the matrix comprises Collagen I.

9. The method of claim 1, wherein the matrix comprises hyaluronate.

10. The method of claim 1, wherein the matrix comprises nanofibers.

11. The method of claim 1, wherein the matrix comprises a repeating peptide sequence.

12. The method of claim 11, wherein the repeating peptide sequence is RADA.

13. The method of claim 1, comprising culturing the fibroblasts under conditions such that at least some of the fibroblasts form non-fibroblast multipotent cells.

14. A cell culture, comprising a three-dimensional matrix seeded with fibroblasts, and further comprising an anti-inflammatory factor.

15. The cell culture of claim 14, wherein the three-dimensional matrix comprises a self-assembled peptide.

16. The article of claim 14, wherein the culture is in vitro.

17. A three-dimensional matrix containing an anti-inflammatory factor.

18. The matrix of claim 17, wherein the three-dimensional matrix further comprises fibroblasts.

19. A method for promoting wound healing, comprising:
inserting, into a wound of a subject, a three-dimensional matrix containing an anti-inflammatory factor in an effective amount to promote wound healing.

20. The method of claim 19, further comprising limiting exposure of the wound to gaseous oxygen.

21. The method of claim 20, wherein the step of limiting exposure of the wound to gaseous oxygen comprises applying a membrane at least substantially impermeable to oxygen to at least a portion of the wound.

22. The method of claim 19, further comprising applying an anticoagulant to the subject.

23. The method of claim 19, wherein the wound is a skin wound.

24. The method of claim 19, wherein the wound is a burn.

25. The method of claim 19, wherein the wound is a severed digit.

26. The method of claim 19, wherein the three-dimensional matrix is seeded with cells.

27. The method of claim 26, wherein at least some of the cells are fibroblasts.

28. The method of claim 27, wherein the fibroblasts are isolated from the subject having the wound.

29. A method for promoting wound healing, comprising:
inserting, into a wound, a three-dimensional matrix; and suppressing an immune response within the wound in an effective amount to promote wound healing.

30. The method of claim 29, further comprising immobilizing the three-dimensional matrix in the wound with a clamp.

31. A method of producing non-fibroblast multipotent cells, comprising:
culturing fibroblasts within a three-dimensional matrix under conditions such that at least some of the fibroblasts form non-fibroblast multipotent cells.

32. The method of claim 31, wherein the method is performed in vitro.

33. The method of claim 31, further comprising isolating at least some of the non-fibroblast cells from the 3-dimensional matrix.

34. The method of claim 31, wherein at least some of the non-fibroblast cells are able to differentiate into more than one type of cell.

35. The method of claim 31, wherein at least some of the non-fibroblast multipotent cells are progenitor-like cells.

36. The method of claim 31, wherein at least some of the non-fibroblast multipotent cells are able to differentiate into more than one cell type.

37. The method of claim 31, wherein at least some of the non-fibroblast cells are osteoblast-like cells.

38. The method of claim 31, wherein at least some of the non-fibroblast cells form a mineralized matrix.

39. The method of claim 31, wherein at least some of the non-fibroblast cells express alkaline phosphatase activity.

40. The method of claim 31, wherein at least some of the non-fibroblast cells express collagen I.

41. The method of claim 31, wherein at least some of the non-fibroblast cells exhibit intracellular osteopontin.

42. The method of claim 31, wherein at least some of the non-fibroblast cells express transcription factor Runx2.

43. A method for promoting wound healing, comprising:
cauterizing at least one artery within a wound; and inserting, into the wound, a three-dimensional matrix in an effective amount to promote wound healing.

44. A method for promoting tissue growth, comprising:
removing a tissue comprising fibroblasts from a subject;
extracting fibroblasts from the tissue;
adding the fibroblasts to a three-dimensional matrix; and
implanting the three-dimensional matrix into the subject in an effective amount to promote tissue growth.

45. The method of claim 44, wherein the fibroblasts are implanted into the subject within 1 day after removal of the tissue from the subject.

46. The method of claim 44, wherein the fibroblasts are grown within the three-dimensional matrix for at least about a week.

47. The method of claim 44, wherein the three-dimensional matrix is implanted into a wound of the subject.

48. The method of claim 47, wherein the wound is a skin wound.

49. The method of claim 47, wherein the wound is a burn.

50. The method of claim 47, wherein the wound is a severed digit.

51. The method of claim 44, wherein the three-dimensional matrix is a self assembling peptide.

52. The method of claim 31, further comprising implanting at least some of the non-fibroblast multipotent cells into a subject.

53. A method for promoting wound healing, comprising:
implanting fibroblasts into a wound; and
suppressing an immune response within the wound in an effective amount to promote wound healing.

54. A method of regenerating tissue, comprising:
applying a three-dimensional matrix to a severed tissue, the matrix comprising one or more regeneration factors; and
reducing exposure of the severed tissue to oxygen to promote regeneration of the tissue.

55. The method of claim 54, wherein the tissue is severed by a surgical procedure.

56. The method of claim 54, wherein the severed tissue is a severed digit.

57. A method, comprising:
culturing stem cells in a three-dimensional matrix for at least 7 days in media substantially free of stem cell promoting factors; and
thereafter, identifying at least some of the cells as stem cells.

58. The method of claim 57, wherein the act of identifying comprises identifying at least some of the cells using an Oct4 expression assay.

59. The method of claim 57, further comprising causing at least some of the stem cells to form osteoblast-like cells.

60. The method of claim 57, further comprising isolating at least some of the stem cells from the 3-dimensional matrix after culturing the cells.

61. The method of claim 57, further comprising causing at least some of the stem cells to form a differentiated tissue.

62. The method of claim 57, further comprising identifying stem cells within the differentiated tissue.

63. The method of claim 57, wherein the three-dimensional matrix is a self assembling peptide.

64. A method for producing adipose tissue, comprising:
culturing fibroblasts in a three-dimensional matrix composed of a self assembling peptide to produce adipose tissue.

65. The method of claim 64 wherein an adipose specific differentiation factor is not added to the culture.

66. The method of claim 64 wherein the culture is performed in vivo.

67. A method for producing chondrocytes, comprising:
culturing fibroblasts under chondrogenic differentiation conditions in a three-dimensional matrix composed of a self assembling peptide to produce chondrocytes.

68. The method of claim 64 wherein the culture is performed in vivo.


SEQUENCE LISTING (5 sequences)

SEQ ID NO: 1 - protein, 16 aa, Artificial sequence
  Synthetic polypeptide
RADARADARADARADA

SEQ ID NO: 2 - protein, 16 aa, Artificial sequence
  Synthetic polypeptide
AEAEAKAKAEAEAKAK

SEQ ID NO: 3 - protein, 16 aa, Artificial sequence
  Synthetic polypeptide
ARARADADARARADAD

SEQ ID NO: 4 - DNA, 20 bp, Artificial sequence
  Synthetic oligonucleotide
catgtactctttcttgctgg

SEQ ID NO: 5 - DNA, 20 bp, Artificial sequence
  Synthetic oligonucleotide
ggtctcgggaaagcagtggc